(19) United States
(12) Patent Application Publication
(10) Pub. No.: US 2015/0065455 A1
(43) Pub. Date: Mar. 5, 2015

(54) METHODS OF ASSESSING SUSCEPTIBILITY TO DRUG-INDUCED THROMBOCYTOPENIA

(71) Applicants: Herve Watier, Ballan-Mire (FR); Yves Gruel, Tours (FR); Claire Pouplard, Tours (FR)

(72) Inventors: Herve Watier, Ballan-Mire (FR); Yves Gruel, Tours (FR); Claire Pouplard, Tours (FR)

(21) Appl. No.: 14/199,342

(22) Filed: Mar. 6, 2014

Related U.S. Application Data
(63) Continuation of application No. 11/629,808, filed on Dec. 15, 2006, now abandoned.
(60) Provisional application No. 60/580,174, filed on Jun. 15, 2004.

(51) Int. Cl. C12Q 1/68; G01N 33/68

(52) U.S. Cl. CPC C12Q 1/6883; G01N 33/6893; C12Q 2600/106; G01N 2800/52
USPC 514/56; 435/6.11; 435/7.92

(57) ABSTRACT

The invention relates to assessing the FcγRIIIa-158 polymorphism in a subject in order to determine susceptibility of the subject to drug induced thrombocytopenia, as well as therapies and therapeutic compositions based on the use of this biomarker.

### FIELD

The invention relates to assessing the FcγRIIIa-158 polymorphism in a subject in order to determine susceptibility of the subject to drug induced thrombocytopenia, as well as therapies and therapeutic compositions based on the use of this biomarker.

### BACKGROUND

Certain drugs or their metabolites induce antibodies in some individuals which can cause immune platelet destruction. Implicated drugs include heparin, especially unfractionated heparin, GPIIb/IIIa inhibitors such as abciximab (ReoPro), xemilofiban, orbofiban eptifibatide (Integrilin) and tirofiban (Aggrastat), quinidine and quinine (stereoisomers of each other), sulfonamide antibiotics and many others (Pouplard, et al, (1999) Circulation 99-2530-2536; Waldmann et al, (2000) Hematology 394-406; R. H. Aster, in Platelet Immunobiology: Molecular and Clinical Aspects. T. J. Kunicki and J. N. George eds., Lippincott, Philadelphia, pp. 387-435, 1989; N. R. Shulman, et al., “Platelet Immunology” in Hemostasis and Thrombosis: Basic Principles and Clinical Practice. R. W. Culman, J. Hirsh, V. J. Marder, E. W. Salzman, eds. Lippincott, Philadelphia, 2nd ed., pp. 452-529, 1989).

A few of these drugs, such as penicillin, appear to bind covalently to platelet proteins and stimulate the formation of antibodies specific for the drug-protein complex (hapten-dependent antibodies) (D. J. Salamon, et al., Transfusion 24:395, 1984). More often, however, the sensitizing drug or one of its metabolites induces the formation of antibody by an unknown mechanism (Aster, supra, 1989; A. Salama, et al, Sem. Hematol. 29:54-63, 1992). The resulting antibodies bind to platelets only in the presence of drug to cause platelet destruction. Evidence (D. J. Christie, et al., J. Clin. Invest. 75:310, 1985; D. J. Christie, et al., J. Clin. Invest. 70:989, 1982) and others (C. Mueller-Eckhardt, et al., Trans. Med. Rev. 4:69, 1990; A. Salama, et al., Semin. Hematol. 29:54, 1992) indicates that in such cases, the drug binds non-covalently and reversibly to selected platelet membrane proteins to induce conformational changes or form compound epitopes that are recognized by the antibodies. Drug-dependent binding of the antibodies to platelets causes the platelets to be destroyed. In the several forms of drug-induced immune thrombocytopenia, platelet counts are often very low and bleeding complications are frequently severe.

One type of drug-induced thrombocytopenia (DIT) has emerged with the advent of novel therapies for the treatments to block platelet aggregation. This DIT is caused by inhibitors of platelet glycoprotein IIb/IIIa (GPIIb/GPIIIa). These drugs have, upon analysis from large therapeutic trials or ongoing treatment, been found to induce thrombocytopenia, including acute severe thrombocytopenia. When drugs of the fiban type such as xemilofiban and orbofiban were used, fiban-dependent antibodies were identified as the major cause of acute severe thrombocytopenia (Brassard et al, Thromb. Haemost. 2002, 88(6):892-7). However, such DIT is not limited to fiban drugs, and was more recently observed with a range of GPIIb/GPIIIa inhibitors, including abciximab (ReoPro) which is an monoclonal antibody-based drug as well as eptifibatide (Integrilin) and tirofiban (Aggrastat) (Abrams and Cines, Cur. Hematol. Rep. 2004, 3(2):143-7.

Another type of drug-induced thrombocytopenia, known for many years, is called heparin-induced thrombocytopenia (HIT) which occurs in patients treated with heparin to prevent or treat thrombosis. Heparin is a family of polysaccharide species consisting of chains made up of alternating, 1-4 linked and variously sulfated residues of glucuronic acid or iduronic acid and D-glucosamine. (B. Casu, “Methods of structural analysis” in Heparin: Chemical and Biological Properties, Cinical Applications, D. A. Lane and U. Lindahl, eds. CRC Press, Inc. Boca Raton, Fla., 1989, pp. 25-49.) In man and animal species, heparin is normally found in storage granules of mast cells (tissue basophils) (L. Enerback, “The mast cell system.” In Heparin: Chemical and biological properties, clinical applications, D. A. Lane and U. Lindahl eds. CRC press, Inc., Boca Raton, Fla., pp. 97-114, 1989). Heparin-like molecules, such as heparan sulfate and chondroitin sulfate are expressed on the surface of endothelial cells that coat the luminal surface of blood vessels and in other tissues where they are coupled to a protein backbone (syndecan) to form a class of molecules known as proteoglycans (Ihrke, et al., Immunology Today 14:500-505, 1993). The heparin-like residues on endothelial cell proteoglycans are thought to provide one means by which abnormal clotting is prevented, allowing the circulating blood to remain in a fluid state (J. A. Marcum, et al., “The biochemistry, cell biology, and pathophysiology of anti-thromboticly active heparin-like molecules of the vessel wall” in Heparin: Clinical and Biological Properties, Clinical Applications. D. A Lane and U. Lindahl eds., CRC Press, Inc., Boca Raton, Fla., pp. 275-294, 1989). Heparin acts as an anti-thrombotic by binding to a co-factor protein, antithrombin III, in such a way as to enable this protein to inhibit certain activated clotting factors, especially activated Factor X (Xa) and thrombin (IIa) (I. Bjork, et al., “Molecular mechanisms of the accelerating effect of heparin on the reactions between antithrombin and clotting proteases” in Heparin: Chemical and Biological Properties, Clinical Applications, D. A Lane and U. Lindahl eds., CRC Press, Inc., Boca Raton, Fla., pp. 229-255, 1989). Heparin of bovine origin appears to be more likely to cause HIT than heparin of porcine origin (W. R. Bell, et al., N. Engl. J. Med. 33:902, 1980).

Thrombocytopenia in patients with HIT is usually not severe enough to result in bleeding. However, patients with HIT often experience thrombosis in major arteries and/or veins which can be fatal or cause the loss of a limb or a stroke. After discontinuation of heparin in patients with HIT, the platelet levels generally return to normal.

HIT appears to be caused by IgG, IgM or IgA antibodies that develop after treatment with heparin (Pouplard, et al, Circulation 99:2530-2536, 1999, and G. P. Visentin, et al., J. Clin. Invest. 93:81-88, 1994 and J. S. Sub, et al, Am J. Hematol, in press, 1995). In the presence of optimal concentrations of heparin, the antibodies activate blood platelets, causing the platelets to release the contents of their storage granules and to undergo membrane changes that create sites for the binding of a coagulation factor, fibrinogen, normally present in plasma (B. H. Chong, et al., Br. J. Haematol. 64:347, 1986). It has been shown that antibodies associated with HIT are specific for complexes of heparin and platelet factor 4 (PF4), a basic heparin-binding protein normally present in platelet storage granules (Visentin, et al., 1994, supra; Amiral, et al., Thromb. Haemostasis 68:95-96, 1992).

Because of the morbidity and mortality associated with DIT, it is important to avoid treatment of a susceptible patient with a drug that can cause DIT. Likewise, it is important that the diagnosis be made quickly and accurately in a patient who develops thrombocytopenia while receiving a drug. Failure to make a diagnosis in such patients can lead to continuation of the drug in question, and possibly a fatal outcome.

Currently available assays include assays used to diagnose drug-induced thrombocytopenia, i.e., binding of IgG or IgM antibodies to normal target platelets in the presence of drug (R. H. Aster, The Immunologic Thrombocytopenias in Platelet Immunology. T. J. Kunicki and J. N. George eds., Lippincott, Philadelphia, Pa., pp. 387-435, 1989) as well as assays to detect complexes of heparin and platelet factor 4 (PF4), a basic heparin-binding protein normally present in platelet storage granules (Visentin, et al., 1994, supra; Amiral, et al., Thromb. Haemostasis 68:95-96, 1992). These assays do not, however, allow prediction of susceptibility in advance of heparin administration or independently of antibody production; a genotyping assay capable of predicting susceptibility to drug-induced thrombocytopenia would therefore have great utility.

### SUMMARY OF THE INVENTION

FcγRIIIa, which is expressed on macrophages and natural killer cells, is encoded by FCGR3A and displays a functional G559T polymorphism, resulting in either a phenylalanine (r) or a valine (V) at amino acid position 158 (Koene et al, Blood. 1997; 90:1109-1114; and Wu et al, J Clin Invest. 1997; 100:1059-1070). Since IgG1 and IgG3, the main IgG subclasses of anti-H/PF4 antibodies, bind more strongly to the FcγRIIIa-158V allotype (Koene, 1997), we hypothesized that the FCGR3A V allele might be associated with drug induced thrombocytopenia conditions such as HIT.

FcγRIIIa-131 and FcγRIIIa-158 genotypes were determined in 102 patients with definite HIT and in 2 control groups of patients treated by heparin (86 subjects without detectable Abs to H/PF4, Ab− group; 84 patients with Abs to H/PF4 without HIT, Ab+ group). There were no significant differences in genotype distribution or allele frequencies between the 3 groups for FcγRIIIa-131H/R polymorphism. In contrast, FcγRIIIa-158V homozygotes were more frequent in the HIT group than in the Ab+ group (p=0.02), a difference which was more pronounced in patients with high levels of anti-H/PF4 Abs (p=0.01). Since anti-H/PF4 Abs are mainly IgG1 and IgG3, it was hypothesized that clearance of sensitized platelets may be increased in patients homozygous for the FcγRIIIa-158V allotype, thus contributing to the development of thrombocytopenia.

Thrombocytopenia is associated with platelet activation mediated by FcγRIIa, the only IgG Fc receptor present on platelets, which in the example of HIT, is cross-linked to H/PF4-IgG immune complexes (Chong et al, Br J Haematol. 1989; 73:235-240). In addition, Fc-mediated clearance of platelets involving FcγRIIIa-bearing phagocytic cells could also contribute to thrombocytopenia since HIT IgG may also bind to platelets and accumulate on the cell surface via F(ab)′ domains (Home et al, J Lab Clin Med. 1996; 127:435-442), thus contributing in part to increased platelet-associated IgG in most patients with DIT and HIT (Cines et al, N Engl J. Med. 1980, 303:788-795; Keton et al, J Lab Clin Med. 1984; 103:606-612; and Leroy et al, 1985; 11:326-329).

Accordingly, the present invention establishes, for the first time, an association between the FCGR3A genotype and drug-induced thrombocytopenia. The invention thus provides a marker that can be used to monitor, evaluate or predict a patient's response to a drug capable of inducing anti-platelet antibodies that result in the clearance of platelets and in turn contributes to the development of thrombocytopenia.

In a preferred aspect the invention discloses a marker that can be used to assess a patient's susceptibility to drug-induced thrombocytopenia, or to monitor, evaluate or predict a patient's response to a drug capable of inducing antibodies that bind or associate with platelets that result in the clearance of platelets and in turn contributes to the development of thrombocytopenia. In a preferred aspect, the invention provides a marker that can be used to assess a patient's susceptibility to thrombocytopenia induced by an anti-thrombotic composition, for example a composition comprising a GPIIb/IIIa inhibitor, a glycosaminogylan, a heparin, or an analog or derivative thereof. The marker can be used to monitor, evaluate or predict a patient's response to an anti-thrombotic therapy, more preferably a composition comprising a GPIIb/IIIa inhibitor, a glycosaminoglycan, a heparin or a derivative or analog thereof. In one embodiment, the aforementioned anti-thrombotic composition or therapy is a composition or therapy other than heparin, particularly unfractionated heparin.

In a preferred aspect, the marker can be used to assess susceptibility to thrombocytopenia in a patient having antibodies that associate with or bind platelets (anti-platelet antibodies), preferably in a patient having anti-platelet factor 4 (PF4) antibodies, anti-heparin-PF4 (H/PF4) antibodies, or anti-cytokine antibodies, such as anti-IL-8 or anti-NAP-2 antibodies. As described in Amiral et al, Blood 88(2): 410-416 (1996), anti-IL-8 or anti-NAP-2 antibodies may exist in the absence of anti-heparin-PF4 (H/PF4) antibodies, and the former may be pre-existing in a patient (for example due to an underlying inflammatory condition) and can be mobilized by drug treatment.

This invention thus introduces new pharmacogenetic approaches in the management of patients treated with compositions capable of inducing the formation of antibodies that bind to platelets to cause immune-mediated destruction of platelets. In many cases, the antibodies bind to platelets only in the presence of the compositions. The compositions include for example drugs that induce antibodies in a patient against drug-protein complexes, for example as observed with heparin. The knowledge that a patient is susceptible to thrombocytopenia can be used to select a course of treatment for the patient that diminishes or avoids the risk of developing such antibodies. Alternatively, the knowledge that a patient is susceptible to thrombocytopenia can be used to select a course of treatment for the patient that decreases or inhibits the destruction of platelets or that treats thrombocytopenia, prevents thrombocytopenia, or otherwise treats or prevents a biological consequence of thrombocytopenia.

Also provided is the use of a drug known or suspected to be capable of causing drug-induced thrombocytopenia for the manufacture of a medicament, wherein the medicament is administered, or is for administration, to a subject after the FCGR3A158 genotype of said subject has been determined. In another aspect the invention provides the use of a drug known or suspected to be capable of causing drug-induced thrombocytopenia for the manufacture of a medicament for administration to a subject having a valine at position 158 of FcγRIIIa receptor and being determined to have an increased susceptibility to drug-induced thrombocytopenia; preferably the drug is provided at a dosage that is lower than for a subject having a phenylalanine at position 158 of FcγRIIIa receptor and thereby being determined to have a decreased susceptibility to drug-induced thrombocytopenia. Further provided is the use of a drug known or suspected to be capable of causing drug-induced thrombocytopenia for the manufacture of a medicament for administration to a subject having a phenylalanine at position 158 of FcγRIIIa receptor and being determined to have a decreased susceptibility to drug-induced thrombocytopenia; preferably the drug is provided at a dosage that is higher than for a subject having a valine at position 158 of FcγRIIIa receptor and thereby being determined to have an increased susceptibility to drug-induced thrombocytopenia.

### DETAILED DESCRIPTION OF THE FIGURES

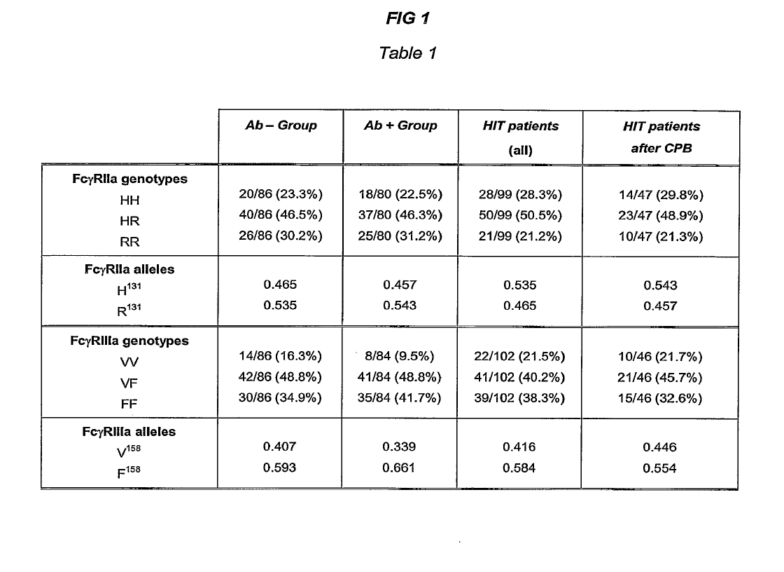

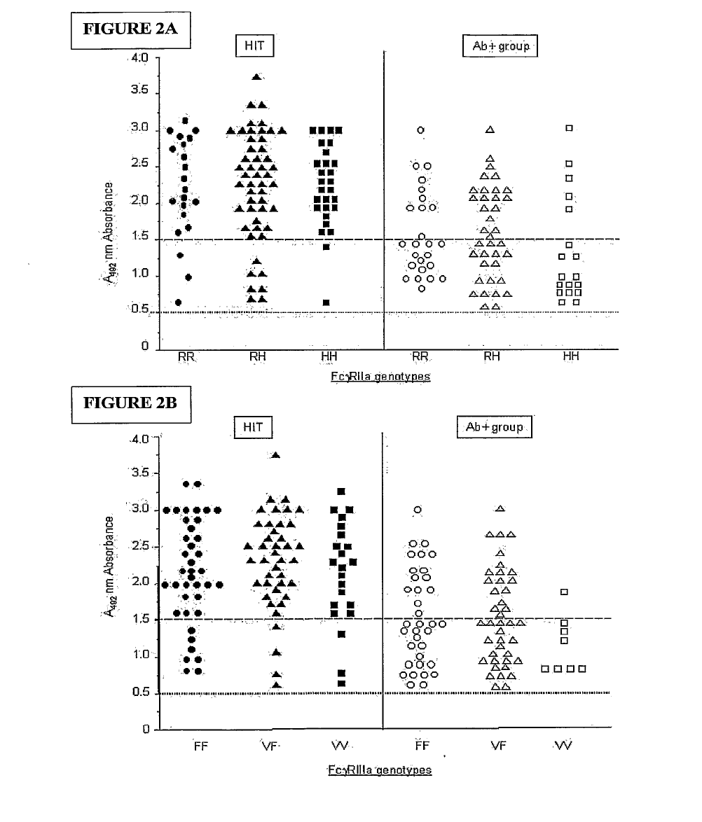

FIG. 1: Table 1 showing FcγRIIa-131 and FcγRIIIa-158 genotypes and allele frequencies in control subjects, all HIT patients and those who had undergone cardiopulmonary bypass (CPB).

FIG. 2: FcγRIIa (A) and FcγRIIIa (B) genotypes in HIT patients and Ab+ group, according to levels of anti-H/PF4 antibodies measured by ELISA.

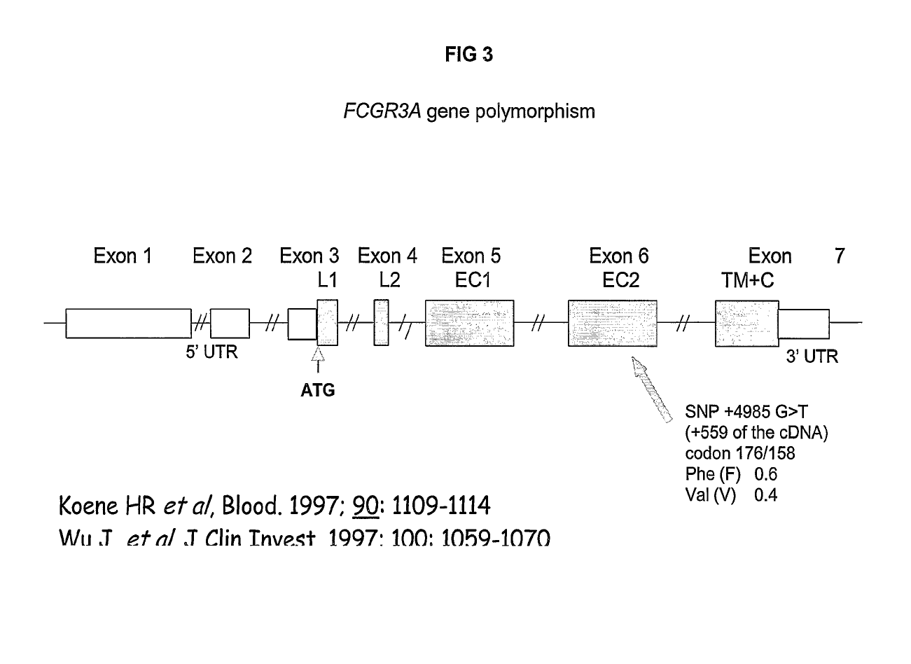

FIG. 3: Genetic organization of the human FCGR3A gene

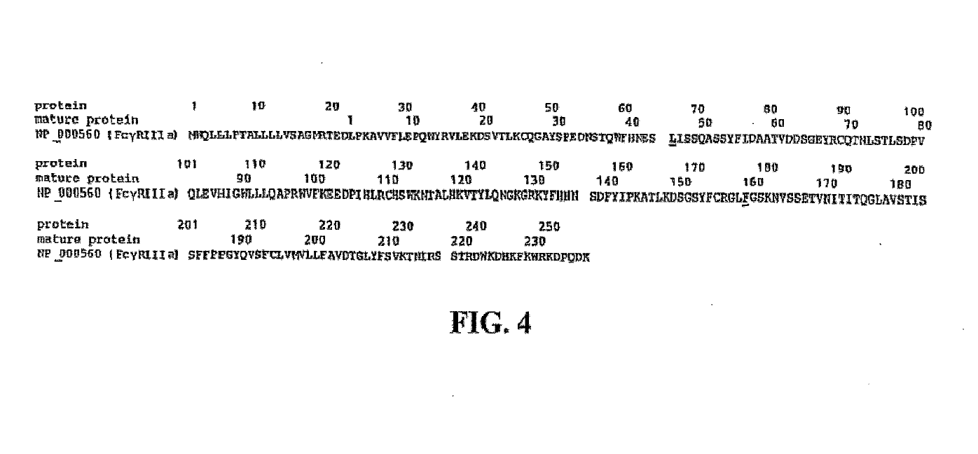

FIG. 4: Amino acid sequences of human FcγRIIIa-158F (SEQ ID NO:7)

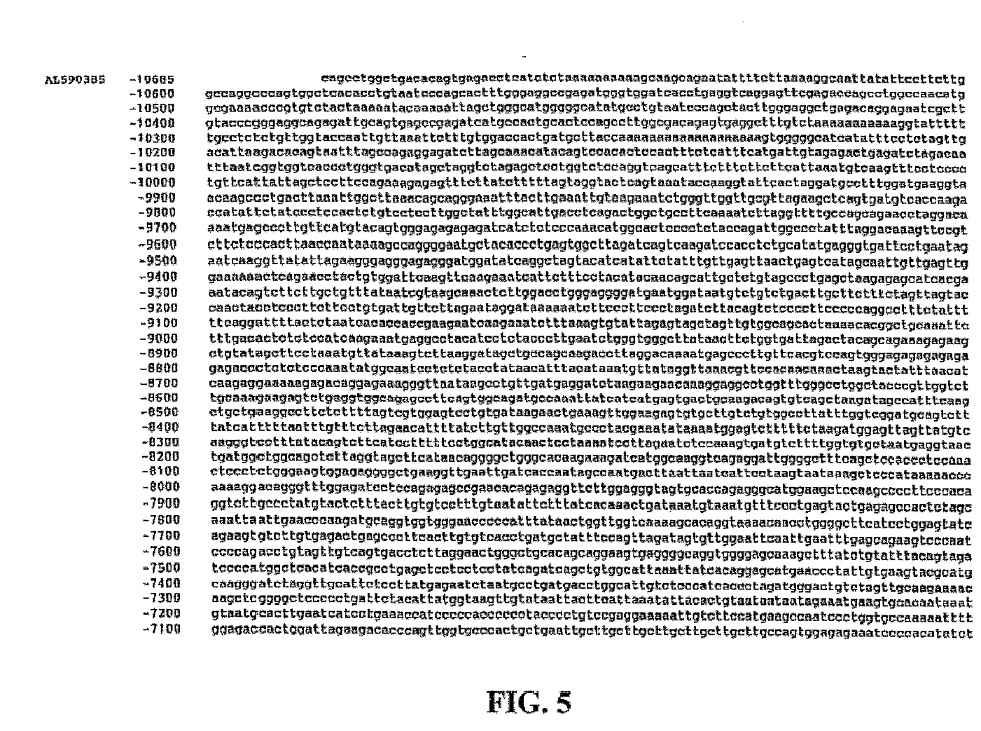

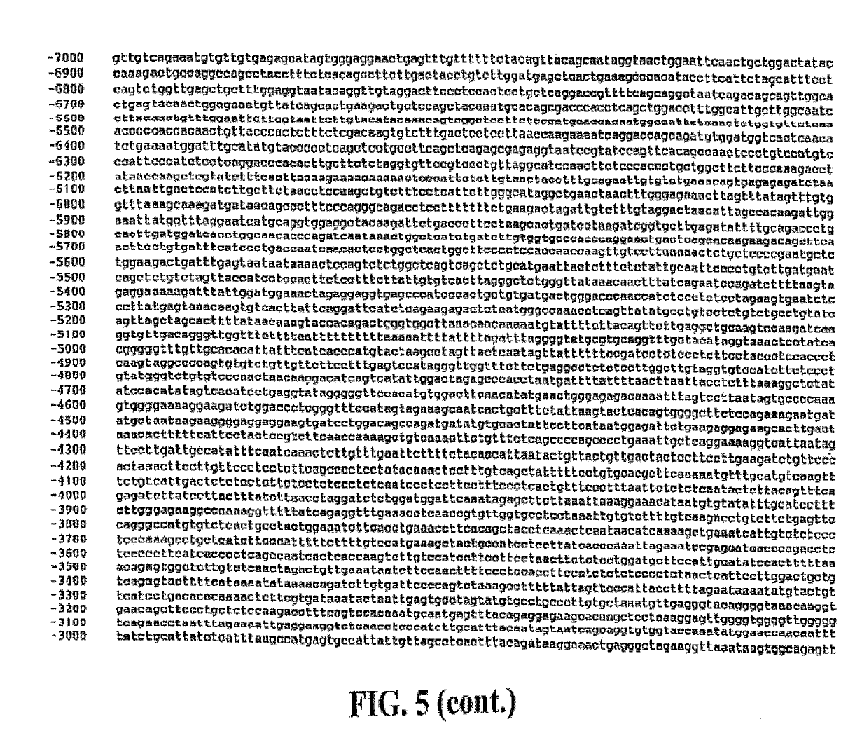

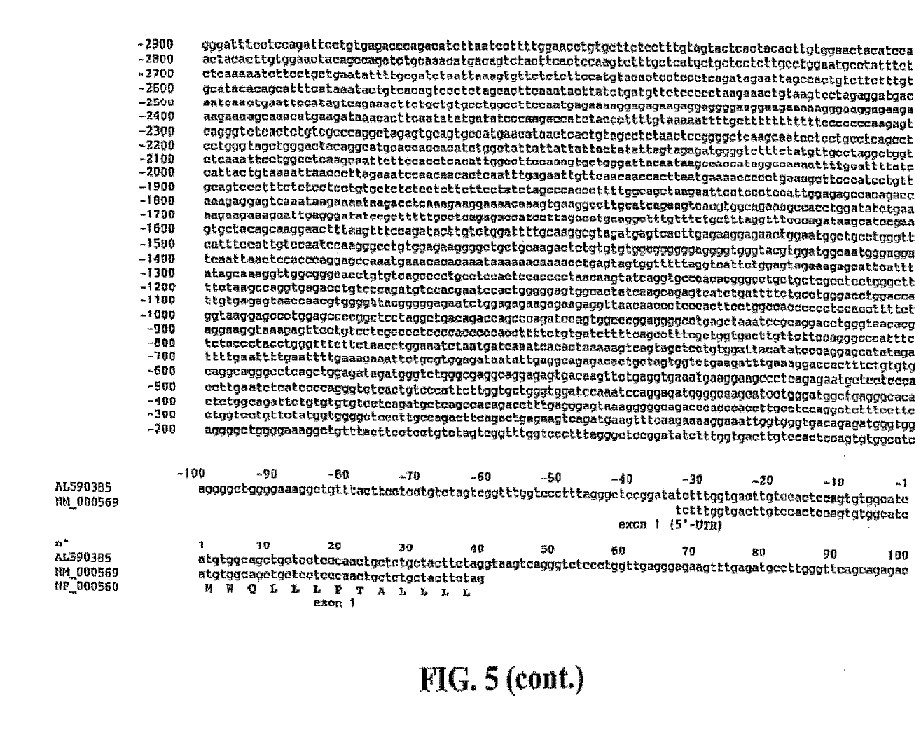

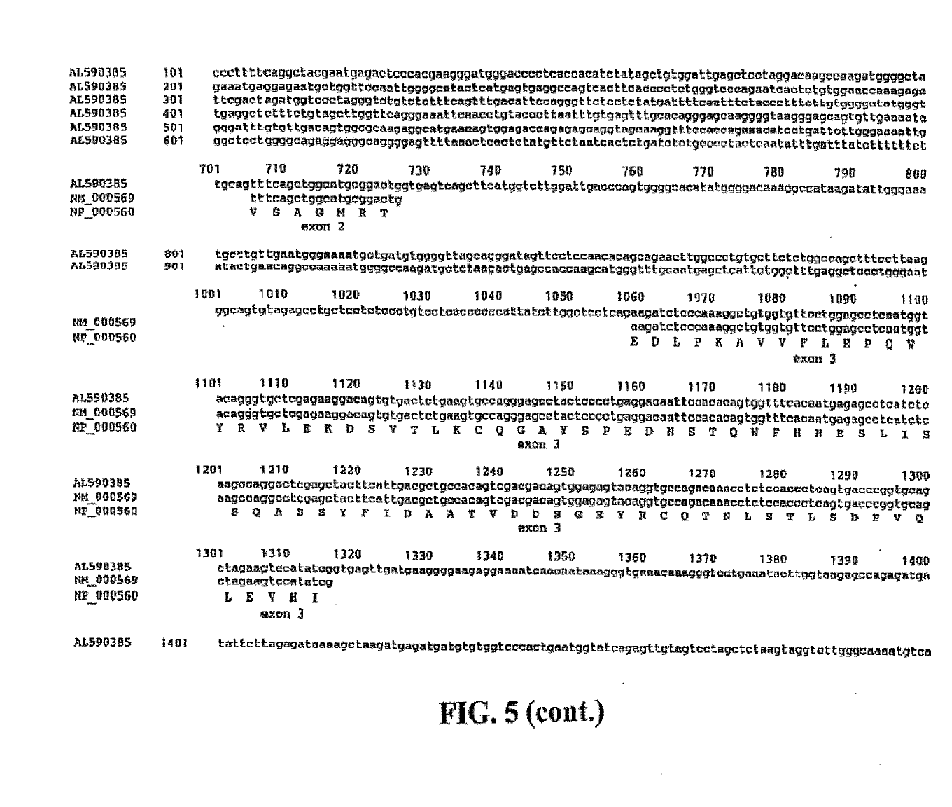

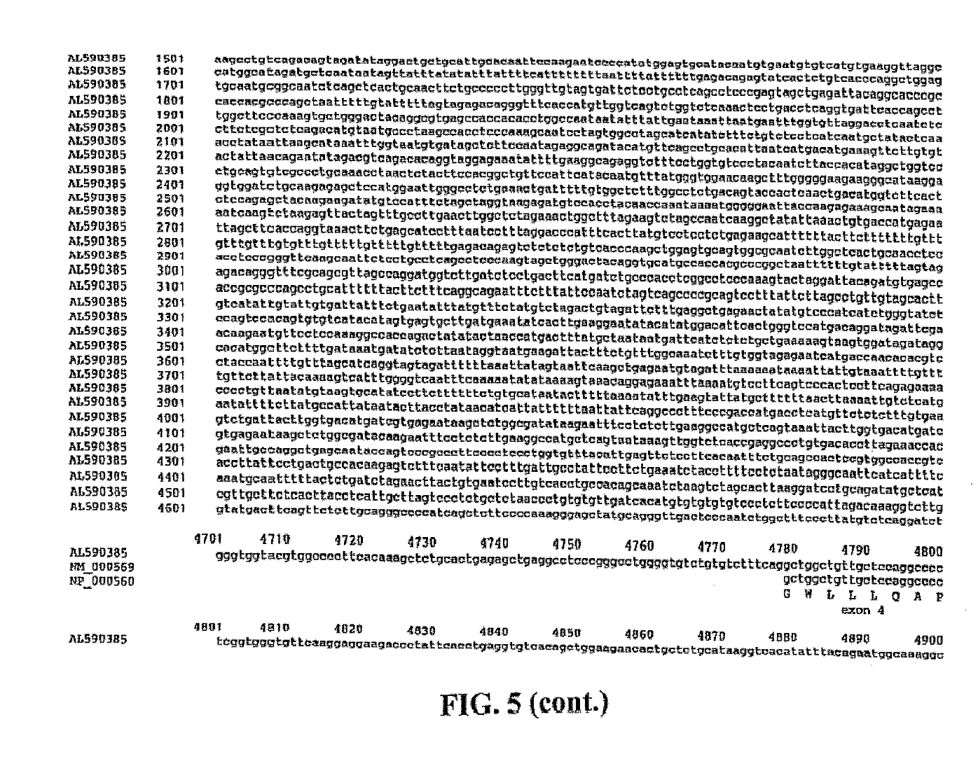

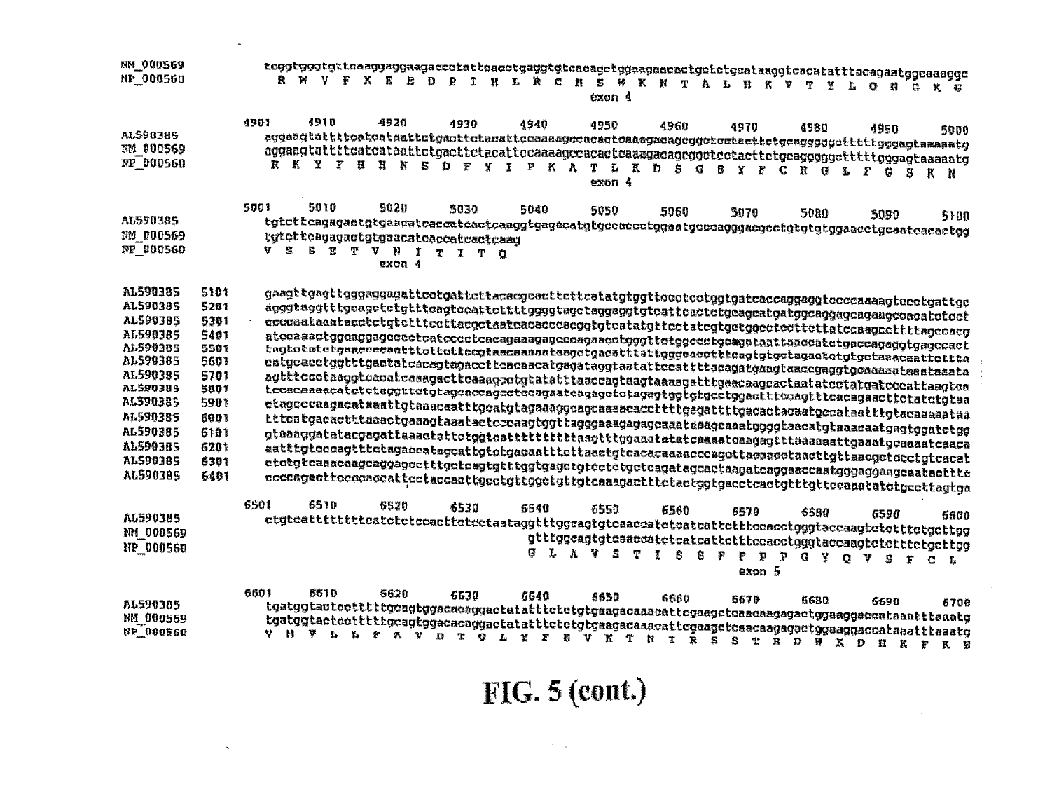

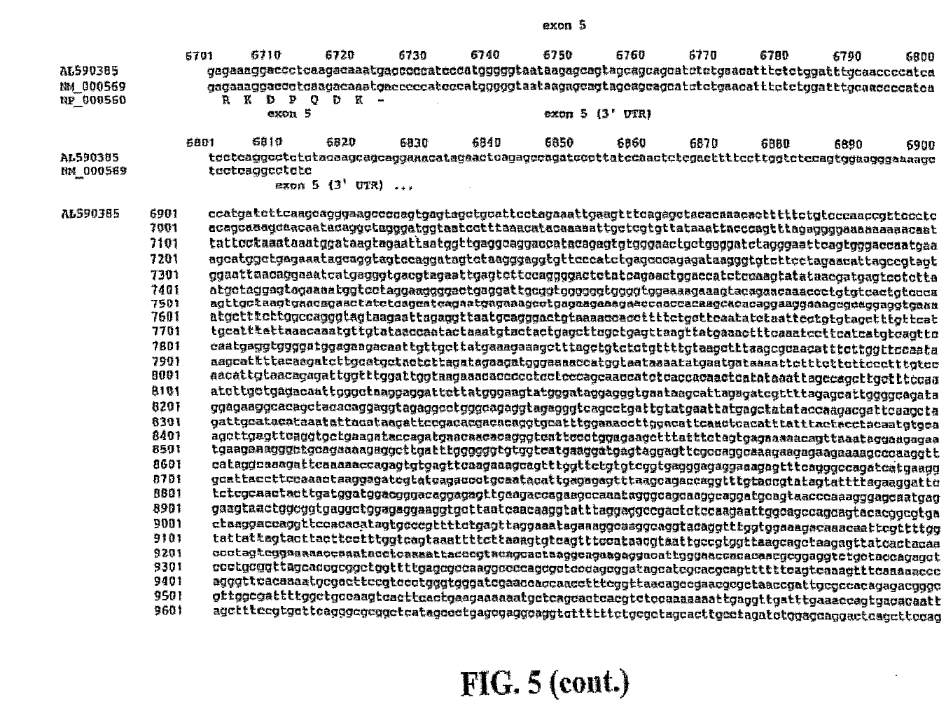

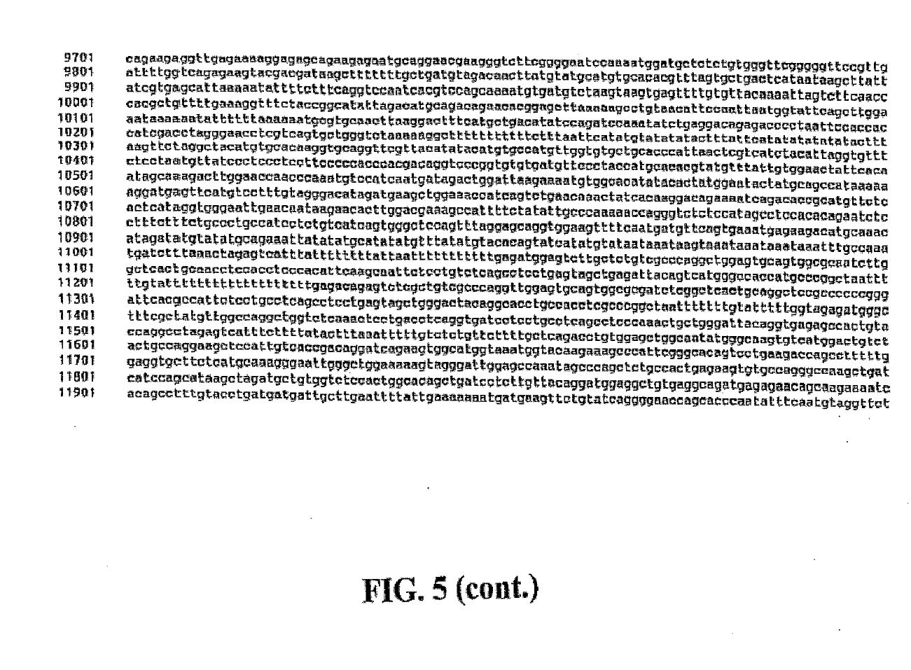

FIG. 5: Nucleic acid sequence of human FCGR3A-158F (SEQ ID NO:8)

### DETAILED DESCRIPTION

There are a number of biological consequences of thrombocytopenia. The main effect of a reduced platelet count in thrombocytopenia is an increased risk of bleeding, although this rarely occurs until there are less than 80-100 million platelets per ml (×10⁹/L). There is not a close relationship between the number of platelets and the severity of bleeding, but there is an increasing risk of hemorrhage if platelet numbers fall or if platelet function is impaired. There is a particularly high risk of spontaneous bleeding once the platelet count drops below 10 million per ml. The bleeding is usually seen on the skin in the form of tiny pin-prick hemorrhages (purpura), or bruises (ecchymoses) following minor trauma. Bleeding from the nose and the gurus is also quite common. More serious haemorrhage can occur at the back of the eye (retina), sometimes threatening sight. But the most serious complication, which is potentially fatal, is spontaneous bleeding inside the head (intracranial) or from the lining of the gut (gastrointestinal).

However, biological consequences of DIT and especially HIT may also include, but are not limited to thrombosis, venous and arterial thrombosis, particularly limb deep venous thrombosis, pulmonary embolism and the most common consequences of thrombosis such as stroke and myocardial infarction. Examples associated particularly with HIT are described for example in Wartenkin, Arch. Pathol. Lab. Med. 126:1415-1423 (2002) the disclosure of which is incorporated herein by reference.

A preferred object of this invention is a method of assessing the susceptibility of a patient to drug-induced thrombocytopenia, the method comprising determining in vitro the FCGR3A genotype and/or the presence of a polymorphism in the FcγRIIIa receptor of said subject. In preferred aspects, the invention provides a method of assessing the susceptibility of a subject to heparin-induced thrombocytopenia and GPIIb/IIIa-inhibitor induced thrombocytopenia, comprising determining in vitro the FCGR3A genotype and/or the presence of a polymorphism in the FcγRIIIa receptor of said subject. More specifically, the method comprises determining in vitro the FCGR3A158 genotype of said subject.

A further object of this invention is a method of selecting patients for treatment with a drug, preferably an anti-coagulant, anti-thrombotic, heparin-based, GPIIa/IIIb inhibitor or antibiotic composition, the method comprising determining in vitro the FCGR3A genotype and/or the presence of a polymorphism in the FcγRIIIa receptor of said subject. More specifically, the method comprises determining in vitro the FCGR3A 158 genotype of said subject. One preferred object of this invention is a method of improving the efficacy or treatment condition or protocol of an anti-thrombotic or anti-coagulant composition in a subject, comprising determining in vitro the FCGR3A genotype and/or the presence of a polymorphism in the FcγRIIIa receptor of said subject. More specifically, the method comprises determining in vitro the FCGR3A 158 genotype of said subject.

More specifically, determining in vitro the FCGR3A158 genotype of a subject comprises determining the amino acid residue present at position 158 of FcγRIIIa receptor (or corresponding codon in the FCGR3A gene), wherein the determination that a subject has for a valine at position 158 is indicative of an increased susceptibility to DIT, and the determination that the subject has a phenylalanine at position 158 is indicative of a decreased susceptibility to DIT

The methods of the invention can be used particularly advantageously in methods of diagnostics, prognostics and treatment. Preferably, said genotype is indicative of the consequences of a therapy. In one example, the methods of the invention are used to determine the amount and administration regimen of a therapeutic composition to be administered to a subject. In another example, the methods of the invention are used to select a therapeutic composition to be administered to a subject—for example therapeutic compositions that are less likely to induce drug-induced thrombocytopenia. In other aspects, the methods are useful in the testing and especially in clinical trials of anti-thrombotic or antibiotic compositions, or any other compositions known or suspected of being capable of inducing thrombocytopenia or antibodies that bind or associate with platelets.

Drug Induced Thrombocytopenia with Heparin (Heparin Induced Thrombocytopenia; HIT)

In the case of HIT, it could be possible that subjects having increased susceptibility to HIT should be treated with anti-thrombotic compositions less likely to induce HIT or anti-platelet antibodies.

In certain embodiments, the present invention provides methods of administering anti-thrombotic therapies, particularly compositions, to a subject so as to inhibit blood coagulation. As used herein, the terms “inhibit” and “inhibiting” in the context of coagulation, mean halting or decreasing the extent of blood coagulation. By “halting or decreasing the extent of blood coagulation,” it is intended that the compositions are used to reduce, preferably by preventing, the degree of blood clot formation as compared with that observed without administration of the composition.

The anti-thrombotic compositions may be administered to a subject in vivo to inhibit blood coagulation in a subject afflicted with or at risk for developing blood clots, or generally to treat any condition for which anti-thrombotic therapy is indicated, particularly in cardiac surgery such as cardiopulmonary bypass, and coronary artery and cerebrovascular disease. Illustrative examples of clinical settings in which the compositions can be used include treatment of myocardial infarction, pulmonary embolism, cerebrovascular disease, and the like. For example, they can be used in the treatment of venous thrombosis and thromboembolic disease, arterial thrombosis and thromboembolic disease, myocardial infarctions, pulmonary embolism, cerebrovascular disease, thrombotic occlusions during and subsequent to thrombolytic therapy or angioplastic therapy and, in general, any other condition for which anti-thrombotic therapy is indicated. Such other conditions include primary and secondary hypercoagulable states (Nachman et al., (1993) Ann. Intern. Med. 119:819), including ATIII and HCII-deficient states (or other serpin deficiencies), and thrombotic complications of other diseases, for example, cancer, tumor metastasis, diabetes, chronic inflammation, sepsis, shock, Disseminated Intravascular Coagulation (DIC), and other conditions where prophylactic anti-thrombotic effects are desired.

Most preferably patients determined to be susceptible to HIT due to their FCGR3A 158 genotype are treated with anti-thrombotic compositions other than unfractionated heparin—it has been reported that unfractionated heparin in particular is associated with HIT and anti-PF4 antibodies to a greater extent that low molecular weight heparin (Pouplard et al, Circulation 99:2530-2536 (1999), the disclosure of which is incorporated herein by reference). Thus, in one aspect, low-molecular weight heparin can be used to treat high-risk subjects. Other exemplary compositions that can be used in place of a compound known or suspected to induce HIT include danaparoid or antithrombin drugs including but not limited to hirudin or melagatran. Other examples of anti-thrombotic compositions include:
compositions that inhibit platelet reactions (including but not limited to compositions that inhibit platelet adhesion, platelet recruitment or block platelet aggregation),
compositions that inhibit coagulation (including but not limited to compositions that prevent thrombin generation or inhibit thrombin activity),
compositions that enhance natural anticoagulant activity (including but not limited to compositions that modulate the protein C pathway), and
compositions that enhance endogenous fibrinolysis (including but not limited to compositions that block type-1 plasminogen activator inhibitor or inhibit procarboxypeptidase B).

Preferred compositions include platelet inhibitor compositions. Examples include GPIIb/IIIa antagonist compositions such as monoclonal antibodies against the GPIIb/IIIa receptor (c7E3-Fab or abciximab, or antibodies in Lefkovits et al, NEJM (1995) 332:1553-1559) and RGD- and KGD-containing peptides. Other compositions are known, including:
compositions that block the initiation of coagulation, such as
compositions that inhibit Factor VIIa/tissue factor complex, such as tissue factor inhibitors, factor VIIa inhibitors or factor VIIa/tissue factor inhibitors
compositions that block thrombin generation, such as Factor IXa inhibitors or Factor Xa inhibitors
compositions that inhibit thrombin, such as active-site inhibitors, and
compositions that enhance endogenous anticoagulant (including but not limited to protein C or activated protein C, soluble thrombomodulin, thrombin variants and allosteric modulators or thrombin.

Other non-limiting examples of anti-thrombotic compositions include those disclosed herein and in Weitz and Hirsch, Chest 114715S-727S (1998), the disclosure of the compositions therein is incorporated herein by reference.

Subjects at high-risk for HIT can be treated with glucosaminoglycan, heparin compositions, unfractionated heparin (UFH) or more preferably analogs or derivatives thereof, at modified dosages or administration regimens that are less likely to induce HIT or anti-platelet antibodies. In other examples, subjects at high-risk for HIT can be treated with glucosaminoglycan, heparin or compositions, or more preferably analogs or derivatives thereof which are modified to decrease the induction of HIT or anti-platelet antibodies compared to unfractionated heparin (UFH) or compared to the analogous compound.

Drug Induced Thrombocytopenia with GPIIb/IIIa Inhibitors

In the case of GPIIb/IIIa inhibitors, subjects having increased susceptibility to DIT can be treated with anti-thrombotic compositions less likely to induce thrombocytopenia or anti-platelet antibodies than inhibitors of platelet aggregation, or more specifically GPIIb/IIIa inhibitors. In the case of GPIIb/IIIa inhibitors, the present invention provides methods of administering anti-thrombotic therapies, particularly compositions, to a subject so as to inhibit blood coagulation.

Most preferably such subjects at risk of DIT are treated with anti-thrombotic compositions other than GPIIb/IIIa inhibitors, or with other GPIIb/IIIa inhibitors that are less likely to cause thrombocytopenia, or induce anti-platelet antibodies. It has been reported that abciximab (ReoPro), xemilofiban, orbofiban eptifibatide (Integrilin) and tirofiban (Aggrastat) can cause DIT and/or can include anti-platelet antibodies. Abciximab (chimeric 7E3 Fab; Reopro) is a Fab fragment of the mouse-human chimeric antibody 7E3 which inhibits ligand binding to the platelet GPIIb/IIIa receptor, the alphaVbeta3 receptor, and one of more activated conformations of the alphaMbeta2 receptor, and approved for use as adjunctive therapy to prevent ischemic complications of percutaneous coronary interventions. Eptifibatide and tirofiban are modelled on the cell recognition sequence arginine-glycine-aspartic acid (RGD) found in several GPIIb/IIIa ligands. Abciximab binds with high affinity (about 1-5 nM). After administration of the recommended bolus dose of 0.25 mg/g, approximately two-thirds of the drug binds rapidly to platelets, resulting in blockage of at least 80% of the GPIIb/IIIa receptors and at least 80% inhibitions of platelet aggregation in response to ADP in a large majority of patients. Similarly, treatment with the recommended infusion of 0.125 ug/kg/min for 12 hours will sustain at least 80% receptor blockage in the majority of patients. Abciximab's pharmacology has several implications, including that patients with severe thrombocytosis need higher doses of antibody to achieve the needed level of GPIIb/IIIa receptor blockage, platelet associated abciximab decreases gradually over time after stopping the drug, and that the amount of unbound antibody in the blood is small permitting the antibody's effect to be reversed rapidly by platelet transfusions. The pharmacokinetics of abciximab are reviewed in Waldmann et al, Hematology (2000), 394-406, the disclosure of which is incorporated herein by reference.

If a subject is found to be susceptible to thrombocytopenia induced by an GPIIb/IIIa inhibitor, the subject can be treated with a different composition. For example exemplary compositions that can be used in place of a GPIIb/IIIa inhibitor known or suspected to induce thrombocytopenia include glucosaminoglycans, heparin compositions, unfractionated heparin (UFH) or more preferably analogs or derivatives thereof, danaparoid or antithrombin drugs including but not limited to hirudin or melagatran. Other examples include:
compositions that inhibit platelet reactions (including but not limited to compositions that inhibit platelet adhesion, platelet recruitment or block platelet aggregation)
compositions that inhibit coagulation (including but not limited to compositions that prevent thrombin generation or inhibit thrombin activity)
compositions that enhance natural anticoagulant activity (including but not limited to compositions that modulate the protein C pathway), and
compositions that enhance endogenous fibrinolysis (including but not limited to compositions that block type-1 plasminogen activator inhibitor or inhibit procarboxypeptidase B).

Other compositions that can be used also include:
compositions that block the initiation of coagulation, such as
compositions that inhibit Factor VIIa/tissue factor complex, such as tissue factor inhibitors, factor VIIa inhibitors or factor VIIa/tissue factor inhibitors
compositions that block thrombin generation, such as Factor Ixa inhibitors or Factor Xa inhibitors
compositions that inhibit thrombin, such as active-site inhibitors, and
compositions that enhance endogenous anticoagulant (including but not limited to protein C or activated protein C, soluble thrombomodulin, thrombin variants and allosteric modulators or thrombin. Other non-limiting examples of anti-thrombotic compositions include those disclosed herein and in Weitz and Hirsch, Chest 114:715S-727S (1998), the disclosure of the compositions therein is incorporated herein by reference.

### Methods of Treatment

The invention also provides a method for monitoring or treating a subject, the method comprising:
determining the FCGR3A genotype in the subject, wherein the genotype is correlated with an increased or decreased likelihood of developing drug-induced thrombocytopenia; and
monitoring said subject for the development of anti-platelet antibodies.

In another aspect, the invention also provides a method for monitoring or treating a subject, the method comprising:
determining the FCGR3A genotype in the subject, wherein the genotype is correlated with an increased or decreased likelihood of developing drug-induced thrombocytopenia; and
monitoring said subject for the development of drug-induced thrombocytopenia or a potential consequence thereof, including but not limited to bleeding or thromobosis, and including but not limited to venous and arterial thrombosis, particularly limb deep venous thrombosis, pulmonary embolism, consequences of thrombosis such as stroke and myocardial infarction.

The invention further provides a method for treating a subject, the method comprising.
determining FCGR3A genotype in the subject, wherein the genotype is correlated with an increased or decreased likelihood of developing drug-induced thrombocytopenia; and
selecting or determining a suitable therapy for treatment of the subject. Preferably the step of selecting or determining a suitable therapy for treatment of the subject comprises selecting a composition to be administered to the subject. In other aspects, the step of or determining a suitable therapy for treatment of the subject comprises selecting a dosage, frequency of administration or duration of treatment with a therapeutic composition to administer to the subject. Preferably the method further comprises (c) administering a therapy, preferably a composition, selected in step (b) to the subject. Preferably, the composition to be administered to a subject is an anti-thrombotic composition.

In another example, the invention discloses a method for treating a subject, the method comprising:
determining FCGR3A genotype in the subject, wherein the genotype is correlated with an increased or decreased likelihood of developing drug-induced thrombocytopenia; and
administering to the subject a suitable therapy for treatment, preferably wherein the therapy is a composition or more preferably an anti-thrombotic composition.

If a subject is determined to have increased susceptibility to DIT, that is, if a subject is has a valine at amino acid residue 158 of the mature FcγRIIIa polypeptide, a composition having lowered tendency to induce DIT or the production of anti-platelet antibodies can be selected and/or administered. For example, in the case of HIT, a number of compositions other than unfractionated heparin as described herein are used to treat subjects at increased risk for developing HIT. In another example, in the case of DIT involving GPIIb/IIIa inhibitors, a number of compositions other than abciximab, xemilofiban, orbofiban eptifibatide (Integrilin) or tirofiban (Aggrastat) are used to treat subjects at increased risk for developing DIT with the respective drug. On the other hand, if a subject is determined to have decreased susceptibility to DIT, that is, if a subject has a phenylalanine at amino acid residue 158 of the mature FcγRIIIa polypeptide, a composition which is known or suspected to have a tendency to induce the production of anti-platelet antibodies or to induce DIT in FcγRIIIa-V subjects can be selected and/or administered. An example of the latter composition is unfractionated heparin or a GPIIb/IIIa inhibitor associated with thrombocytopenia.

In particular, the invention provides methods for treating a subject comprising:
determining the FCGR3A genotype in the subject, wherein the genotype is correlated with an increased or decreased likelihood of having an increased or decreased positive therapeutic response to a composition associated with drug-induced thrombocytopenia; and
selecting or determining a suitable therapy for treatment of the subject. Preferably the therapeutic response is efficacy, for example efficacy in the prevention of coagulation or thrombosis. Preferably the step of selecting or determining a suitable therapy for treatment of the subject comprises selecting a composition to be administered to the subject. In other aspects, the step of or determining a suitable therapy for treatment of the subject comprises selecting a dosage, frequency of administration or duration of treatment with a therapeutic composition to administer to the subject.

In one example, the determination that a subject has for a valine at position 158 of the mature FcγRIIIa polypeptide is indicative of an increased susceptibility to drug-induced thrombocytopenia, and the determination that the subject has a phenylalanine at position 158 of the mature FcγRIIIa polypeptide is indicative of a decreased susceptibility to drug-induced thrombocytopenia, and a patient having increased susceptibility to drug-induced thrombocytopenia is treated with a standard dose, or preferably lower than standard dose of the drug which is known to cause drug-induced thrombocytopenia. In another aspect, a patient having decreased susceptibility to drug-induced thrombocytopenia is treated with a standard or preferably a higher than standard dose of the drug which is known to cause drug-induced thrombocytopenia. The standard dosage for a given drug is generally-ell known in the art, and is preferably the dose approved by a drug regulatory agency for the treatment of human patients (e.g. U.S. Food and Drug Administration); alternatively a lower or higher than standard dosage may be a dosage that is lower or higher, respectively, than a second approved dosage (e.g. if two or more dosages are available or approved for treatment, such as for different FCG3A genotypes). In one embodiment the standard dose is that listed in the Physician's Desk Reference (PDR, published by Thomson Healthcare, for example PDR 2005, 59^{th} Edition, ISBN: 1563634988 the disclosure of which is incorporated herein by reference in its entirety) for the particular drug and indication. In another embodiment, provided is a method of treating a subject having a valine at position 158 of the mature FcγRIIIa polypeptide and having an increased susceptibility to drug-induced thrombocytopenia with a lower than standard dosage of a drug known to cause DIT, as well as treating a subject having a phenyalanine at position 158 of the mature FcγRIIIa polypeptide and having a decreased susceptibility to drug-induced thrombocytopenia with a higher than standard dosage of a drug known to cause DIT. Likewise, provided is the use of a drug known to cause DIT for the manufacture of a medicament, wherein the drug is provided in a lower dosage for the treatment of a subject having a valine at position 158 of the mature FcγRIIIa polypeptide and having an increased susceptibility to drug-induced thrombocytopenia than for a subject having a phenylalanine at position 158 of the mature FcγRIIIa polypeptide and having a decreased susceptibility to drug-induced thrombocytopenia. Also provided is the use of a drug known to cause DIT for the manufacture of a medicament, wherein the drug is provided in a higher dosage for the treatment of a subject having a phenylalanine at position 158 of the mature FcγRIIIa polypeptide and having a decreased susceptibility to drug-induced thrombocytopenia than for a subject having a valine at position 158 of the mature FcγRIIIa polypeptide and having an increased susceptibility to drug-induced thrombocytopenia. In preferred embodiments, the drug known to cause DIT is selected from the group consisting of heparin, GPIIb/IIIa inhibitors such as abciximab (ReoPro), xemilofiban, orbofiban, eptifibatide (Integrilin) and tirofiban (Aggrastat), quinidine, quinine, sulfonamide antibiotics, and derivatives and analogs thereof and generally compounds structurally related thereto.

In particular, the invention provides methods for treating a subject comprising:
determining the FCGR3A genotype in the subject, wherein the genotype is correlated with an increased or decreased likelihood of having an increased or decreased positive therapeutic response to an anti-thrombotic composition; and
selecting or determining an effective amount of said agent to administer to said subject.

Preferably the therapeutic response is efficacy, for example efficacy in the prevention of coagulation or thrombosis. Preferably the step of selecting or determining a suitable therapy for treatment of the subject comprises selecting a composition to be administered to the subject. In other aspects, the step of or determining a suitable therapy for treatment of the subject comprises selecting a dosage, frequency of administration or duration of treatment with a therapeutic composition to administer to the subject.

In another aspect, determining whether a subject has increased or decreased susceptibility to DIT or HIT can be used in a method where a patient at risk of DIT or HIT is administered a treatment for prevention of DIT or HIT, for treatment of DIT or HIT or for treatment of a biological consequence of DIT or HIT. For example, a subject may be administered a composition for the prevention or treatment of bleeding, thrombosis, stroke or myocardial infarct, for the prevention or inhibition of anti-platelet antibody formation, for example inhibition of formation of anti-H/PF4 antibodies, or for the prevention or inhibition of platelet clearance, particularly by NK cells expressing FcγRIIIa, for example by treatment with composition comprising a soluble FcγRIIIa or a monoclonal antibody against FcγRIIIa

Treating and Preventing DIT with FcγRIIIa Binding Proteins

The inventors' discovery showing that FcγRIIIa genotype has an important contribution to thrombocytopenia, including but not limited to drug-induced thrombocytopenia and HIT also provides improved therapeutic approaches. The present invention provides method of treating a subject suffering or susceptible to thrombocytopenia or for preventing thrombocytopenia, comprising administering a composition in a therapeutically effective amount so as to interfere with FcγRIIIa binding to antibodies associated with platelets, preferably so as to interfere with FcγRIIIa-mediated clearance of platelets. This can serve to treat or prevent DIT. Example of compositions that interfere with FcγRIIIa binding to antibodies associated with platelets include but are not limited to FcγRIIIa binding proteins such as soluble FcγRIIIa and fragments or variants thereof, or anti FcγRIIIa antibodies (examples are described in PCT Publication no. WO/03101485, the disclosure of which is incorporated herein by reference). In a preferred example the invention comprises a method for treating a subject comprising: (a) administering to the subject a compound associated with thrombocytopenia, or a compound known or suspected to be capable of inducing thrombocytopenia and (b) administering to the subject a therapeutically effective amount of a FcγRIIIa binding protein. The FcγRIIIa binding protein may be administered before or conjointly with the compound known or suspected to be capable of inducing thrombocytopenia, or may be administered only when a subject is observed to develop anti-platelet antibodies or upon the onset of thrombocytopenia. Examples of compounds known or suspected to be capable of inducing thrombocytopenia include but are not limited to the GPIIb/IIIa inhibitors abciximab, xemilofiban, orbofiban eptifibatide (Integrilin) or tirofiban (Aggrastat), as well as heparin.

In a preferred embodiment, the method comprises (a) determining the FCGR3A genotype in the subject, (b) administering to the subject a compound associated with thrombocytopenia, or a compound known or suspected to be capable of inducing thrombocytopenia, and (c) if it is determined that the subject is susceptible to drug-induced thrombocytopenia, administering to the subject a therapeutically effective amount of a FcγRIIIa binding protein.

### Test-Composition Assessment and Clinical Trials

The methods of the invention can also be used advantageously in a clinical trial to assess subjects' susceptibility to drug-induced thrombocytopenia. Such methods are expected to be useful for example to determine whether one, two or more arms of a clinical trial are balanced for the number of subjects having increased or decreased susceptibility to drug-induced thrombocytopenia. The method can also be used to assess a test composition using in vitro assays, or in animals or in human clinical trials so as to design suitable administration regimens suitable for use in human therapeutic applications. The method of the invention can also be used to select subjects for inclusion in a clinical trial. Methods of the invention are expected to be especially useful in clinical trials where efficacy or side effects of a test composition are to be assessed. For example, in a clinical trial, it may be desirable to assess whether a test composition or a method of treatment has a tendency to cause drug-induced thrombocytopenia, or where is it desirable to assess the therapeutic efficacy of a test composition in the case where drug-induced thrombocytopenia could affect therapeutic efficacy. A particularly suitable application for the methods of the invention is in trials where it is desirable to compare whether a test composition has a higher or lower tendency to cause drug-induced thrombocytopenia or to induce anti-platelet antibodies than a second composition.

Use of a method of the invention in a clinical trial is particularly suitable where the test composition, is known or suspected of or suspected to be capable of inducing the formation of antibodies that bind to platelets to cause immune-mediated destruction of platelets, or capable of inducing DIT or more preferably HIT.

Test compositions can generally be of any type, for example an anti-thrombotic, or an antibiotic. Optionally, the test composition is compared to a another composition used for treatment of the same condition in which the test composition is being assessed or a composition which is an analog or derivative of the test composition or which is otherwise related by structure, function or pharmacological effects to the test composition.

In a preferred example, the test composition is an anti-thrombotic composition, preferably heparin or a GPIIb/IIIa inhibitor. In another aspect, the test composition is a composition that is intended for administration conjointly with heparin treatment, before or after heparin treatment. In other aspects, the test composition is an anti-thrombotic composition which can be used as a substitute for heparin or the GPIIb/IIIa inhibitor, or to modify the administration regimen of heparin or the GPIIb/IIIa inhibitor, preferably decreasing the dosage, frequency of administration or length of treatment.

For instance, the methods of the invention can comprise determining in a subject the FCGR3A genotype, wherein said genotype places said subject into a subgroup for treatment or analysis in a clinical trial, or in a subgroup for inclusion in a clinical trial.

In one aspect, the invention provides a method for the clinical testing of a test composition, the method comprising the following steps.
(a) administering a test composition to a plurality of individuals; and
(b) identifying one or a plurality of individuals having a first FCGR3A genotype and one or a plurality of individuals having a second FCGR3A genotype, preferably wherein a first FCGR3A genotype indicates increased susceptibility to DIT and a second FCGR3A genotype indicates decreased susceptibility to DIT. The method may optionally further comprise: (a) assessing the response to said test composition in said individual(s) having a first FCGR3A genotype; and/or (b) assessing the response to said test composition in the individuals having a second FCGR3A genotype. Preferably, the response to said test composition is assessed both in individuals having said first and said second FCGR3A genotype. Preferably said response is assessed separately in said first and second subpopulations of individuals. Assessing the response to said test composition preferably comprises assessing therapeutic efficacy of the medicament. In another aspect, Assessing the response to said test composition preferably comprises assessing side effects of the test composition, including but not limited to anti-platelet antibodies, anti-PF4 antibodies, anti-H/PF4 antibodies, DIT, HIT and biological consequences thereof.

The invention also concerns a method for the clinical testing of a test composition, the method comprising the following steps.
identifying a first population of individuals having a first FCGR3A genotype and a second population of individuals having a second FCGR3A genotype;
administering a test composition to individuals of said first and/or said second population of individuals. In one embodiment, the test composition is administered to individuals of said first population but not to individuals of said second population. In one embodiment, the test composition is administered to individuals of said second population but not to individuals of said first population. In another embodiment, the test composition is administered to the individuals of both said first and said second populations. For example, in order to assess whether a test composition intended to prevent or treat DIT is efficacious, the test composition can be administered to a population of individuals having increased susceptibility to DIT but not to the population having decreased susceptibility to DIT.

In other example the invention further provides a method for screening or assessing therapeutic composition. It may be advantageous to determine the genotype of patients treated with a test composition in order to more accurately assess the likelihood that the compound is associated with DIT, or to assess the nature of the DIT associated with a composition. Preferably said test in done in vivo, in a non-human mammal or in a clinical trial, but in other aspects the method can also be carried out in vivo wherein the subject from whom the biological sample is obtained is genotyped for FCGR3A. Preferably the invention thus also encompasses a method for assessing a test composition, the method comprising:
determining FCGR3A genotype in the subject, wherein the genotype is correlated with an increased or decreased likelihood of developing drug-induced thrombocytopenia; and
determining or assessing whether a therapeutic composition induces the formation of antibodies, especially anti-platelet antibodies, or is associated with DIT or a biological consequence thereof. Optionally the method comprises correlating the FCGR3A genotype of a subject to the occurrence of DIT or a biological consequence thereof. Such a method may be advantageous in the commercial development of therapeutic compositions, for example to identify and design optimal administration regimens of drugs. Optionally the method comprises designing or selecting a suitable administration regimen for the composition, preferably so as to minimize the formation of drug induced antibodies or anti-platelet antibodies, or more preferably to minimize the occurrence or severity of DIT. Examples include but are not limited to determining or selecting a dosage to be used in treatment of subjects, determining or selecting a frequency of administration or duration of treatment to be used in treatment of subjects, determining or selecting a conjoint therapy to be used in treatment of subjects, for example to reduce the formation or deleterious effects of antibodies, to ameliorate thrombocytopenia or a consequence thereof, for example bleeding or thrombosis.

### Methods for Typing Individuals

According to the invention the term FCGR3A gene refers to any nucleic acid molecule encoding a FcγRIIIa polypeptide in a subject. This term includes, in particular, genomic DNA, cDNA, RNA (pre-rRNA, messenger RNA, etc.), etc. or any synthetic nucleic acid comprising all or part of the sequence thereof. Synthetic nucleic acid includes cDNA, prepared from RNAs, and containing at least a portion of a sequence of the FCGR3A genomic DNA as for example one or more introns or a portion containing one or more mutations. Most preferably, the term FCGR3A gene refers to genomic DNA, cDNA or mRNA, typically genomic DNA or mRNA. The FCGR3A gene is preferably a human FCGRIIIa gene or nucleic acid, i.e., comprises the sequence of a nucleic acid encoding all or part of a FcγRIIIa polypeptide having the sequence of human FcγRIIIa polypeptide. Such nucleic acids can be isolated or prepared according to known techniques. For instance, they may be isolated from gene libraries or banks, by hybridization techniques. They can also be genetically or chemically synthesized. The genetic organization of a human FCGRIIIa gene is depicted on FIG. 3. The amino acid sequence of human FcγRIIIa is represented FIG. 4. Amino acid position 158 is numbered from residue 1 of the mature protein, and corresponds to residue 176 of the pre-protein having a signal peptide. The sequence of a wild type FCGR3A gene is represented on FIG. 3 (see also Genbank accession Number AL590385 or NM_{—}000569 for partial sequence).

Within the context of this invention, a portion or part means at least 3 nucleotides (e.g., a codon), preferably at least 9 nucleotides, even more preferably at least 15 nucleotides, and can contain as much as 1000 nucleotides. Such a portion can be obtained by any technique well known in the art, e.g., enzymatic and/or chemical cleavage, chemical synthesis or a combination thereof. The sequence of a portion of a FCGR3A gene encoding amino acid position 158 is represented below, for sake of clarity:

| | |
|---|---|
| cDNA | 540 550 550 570 500 |
| genomic DNA | 4970 4980 4990 5000. |
| 158F allele | tcctacttctgcagggggctttttgggagtaaaaatgtgtcttca |
| | S Y F C R G L F G S K N V S S |
| 158V allele | tcctacttctgcagggggcttgttgggagtaaaaatgtgtcttca |
| | S Y F C R G L V G S K N V S S |

As indicated above, the invention comprises a method of determining in vitro the FCGR3A158 genotype of said subject. This more particularly comprises determining the nature of amino acid residue present (or encoded) at position 158 of the FcγRIIIa polypeptide.

As indicated throughout the present disclosure, the invention comprises methods comprising determining in vitro the FCGR3A158 genotype of a subject. It will be appreciated that in any of the embodiments of the invention referring to determining the FCGR3A genotype, it will be readily possible to determine the genotype by determining the phenotype, that is by determining the identity of the amino acid residue present (or encoded) at position 158 of the FcγRIIIa polypeptide. Thus, determining the FCGR3A genotype can comprise or consist of determining the identity of the amino acid residue present (or encoded) at position 158 of the FcγRIIIa polypeptide.

Hornozygosity for a Valine at position 158 of the FcγRIIIa receptor is indicative of an increased susceptibility to DIT, and a phenylalanine at position 158 of the FcγRIIIa receptor (heteozygous or homozygous) is indicative of a decreased susceptibility to DIT. The impact of the genotype of the FcγRIIIa receptor at position 158 on susceptibility to DIT is thus generally more strongly marked, that is an increased susceptibility to DIT, when the subject is homozygous at position 158 for Valine. However, subjects homozygous or heterozygous for phenylalanine at position 158 are both less susceptible to DIT than subjects homozygous for a Valine at position 158.

Genotyping the FCGR3A gene or corresponding polypeptide in said subject may be achieved by various techniques, comprising analysing the coding nucleic acid molecules or the encoded polypeptide. Analysis may comprise sequencing, migration, electrophoresis, immuno-techniques, amplifications, specific digestions or hybridisations, etc.

In a particular embodiment, determining amino acid residue at position 158 of FcγRIIIa receptor comprises a step of sequencing the FCGR3A receptor gene or RNA or a portion thereof comprising the nucleotides encoding amino acid residue 158.

In an other particular embodiment, determining amino acid residue at position 158 of FcγRIIIa receptor comprises a step of amplifying the FCGR3A receptor gene or RNA or a portion thereof comprising the nucleotides encoding amino acid residue 158. Amplification may be performed by polymerase chain reaction (PCR), such as simple PCR, RT-PCR or nested PCR, for instance, using conventional methods and primers. A preferred genotyping method, including the disclosure of nucleic acid primers, for determining amino acid residue at position 158 of FcγRIIIa receptor is provided in Dall'Ozzo S, Andres C, Bardos P, Watier H, and Thibault G, J Immunol Methods. (2003) 277(1-2):185-92, which disclosure, including but not limited to specific nucleotide sequences disclosed therein, is incorporated herein by reference in its entirety.

In this regard, amplification primers for use in this invention more preferably contain less than about 50 nucleotides even more preferably less than 30 nucleotides, typically less than about 25 or 20 nucleotides. Also, preferred primers usually contain at least 5, preferably at least 8 nucleotides, to ensure specificity. The sequence of the primer can be prepared based on the sequence of the FCGR3A gene, to allow full complementarity therewith, preferably. The probe may be labelled using any known techniques such as radioactivity, fluorescence, enzymatic, chemical, etc. This labeling can use for example Phosphor 32, biotin (16-dUTP), digoxygenin (11-dUTP). It should be understood that the present invention shall not be bound or limited by particular detection or labelling techniques. The primers may further comprise restriction sites to introduce allele-specific restriction sites in the amplified nucleic acids, as disclosed below.

Specific examples of such amplification primers are, for instance, SEQ ID NO: 1-4.

It should be understood that other primers can be designed by the skilled artisan, such as any fragment of the FCGR3A gene, for use in the amplification step and especially a pair of primers comprising a forward sequence and a reverse sequence wherein said primers of said pair hybridize with a region of a FCGR3A gene and allow amplification of at least a portion of the FCGR3A gene containing codon 158. In a preferred embodiment, each pair of primers comprises at least one primer that is complementary, and overlaps with codon 158, and allows to discriminate between 158V (gtt) and 158F (ttt). The amplification conditions may also be adjusted by the skilled person, based on common general knowledge and the guidance contained in the specification.

In a particular embodiment, the method of the present invention thus comprises a PCR amplification of a portion of the FCGR3a mRNA or gDNA with specific oligonucleotide primers, in the cell or in the biological sample, said portion comprising codon 158, and a direct or indirect analysis of PCR products, e.g., by electrophoresis, particularly Denaturing Gel Gradient Electrophoresis (DGGE).

In an other particular embodiment, determining amino acid residue at position 158 of FcγRIIIa receptor comprises a step of allele-specific restriction enzyme digestion. This can be done by using restriction enzymes that cleave the coding sequence of a particular allele (e.g., the 158V allele) and that do not cleave the other allele (e.g., the 158F allele, or vice versa). Where such allele-specific restriction enzyme sites are not present naturally in the sequence, they may be introduced therein artificially, by amplifying the nucleic acid with allele-specific amplification primers containing such a site in their sequence. Upon amplification, determining the presence of an allele may be carried out by analyzing the digestion products, for instance by electrophoresis. This technique also allows to discriminate subjects that are homozygous or heterozygous for the selected allele.

Examples of allele-specific amplification primers include for instance SEQ ID NO: 3. SEQ ID NO:3 introduces the first 3 nucleotides of the NlaIII site (5′-CATG-3′). Cleavage occurs after G. This primer comprises 11 bases that do not hybridise with FCGR3A, that extend the primer in order to facilitate electrophoretic analysis of the amplification products) and 21 bases that hybridise to FCGR3A, except for nucleotide 31 (A) which creates the restriction site.

In a further particular embodiment, determining amino acid residue at position 158 of FcγRIIIa receptor comprises a step of hybridization of the FCGR3A receptor gene or RNA or a portion thereof comprising the nucleotides encoding amino acid residue 158, with a nucleic acid probe specific for the genotype Valine or Phenylalanine, and determining the presence or absence of hybrids.

It should be understood that the above methods can be used either alone or in various combinations. Furthermore, other techniques known to the skilled person may be used as well to determine the FCGR3A158 genotype, such as any method employing amplification (e.g. PCR), specific primers, specific probes, migration, etc., typically quantitative RT-PCR, LCR (Ligase Chain Reaction), TMA (Transcription Mediated Amplification), PCE (an enzyme amplified immunoassay) and bDNA (branched DNA signal amplification) assays.

In a preferred embodiment of this invention, determining amino acid residue at position 158 of FcγRIIIa receptor comprises:
obtaining genomic DNA from a biological sample,
amplifying the FcγRIIIa receptor gene or a portion thereof comprising the nucleotides encoding amino acid residue 158, and
determining amino acid residue at position 158 of said FcγRIIIa receptor gene.

Amplification can be accomplished with any specific technique such as PCR, including nested PCR, using specific primers as described above. In a most preferred embodiment, determining amino acid residue at position 158 is performed by allele-specific restriction enzyme digestion. In that case, the method comprises:
obtaining genomic DNA from a biological sample,
amplifying the FcγRIIIa receptor gene or a portion thereof comprising the nucleotides encoding amino acid residue 158,
introducing an allele-specific restriction site,
digesting the nucleic acids with the enzyme specific for said restriction site and,
analysing the digestion products, i.e., by electrophoresis, the presence of digestion products being indicative of the presence of the allele.

In an other particular embodiment, the genotype is determined by a method comprising: total (or messenger) RNA extraction from cell or biological sample or biological fluid in vitro or ex vivo, optionally cDNA synthesis, (PCR) amplification with FCGR3A-specific oligonucleotide primers, and analysis of PCR products.

The method of this invention may also comprise determining amino acid residue at position 158 of FcγRIIIa receptor directly by sequencing the FcγRIIIa receptor polypeptide or a portion thereof comprising amino acid residue 158 or by using reagents specific for each allele of the FcγRIIIa polypeptide. This can be determined by any suitable technique known to the skilled artisan, including by immuno-assay (ELISA, EIA, RIA, etc.). This can be made using any affinity reagent specific for a FcγRIIIa158 polypeptide, more preferably any antibody or fragment or derivative thereof. In a particular embodiment, the FcγRIIIa158 polypeptide is detected with an anti-FcγRIIIa158 antibody (or a fragment thereof) that discriminates between FcγRIIIa158V and FcγRIIIa158F, more preferably a monoclonal antibody. The antibody (or affinity reagent) may be labelled by any suitable method (radioactivity, fluorescence, enzymatic, chemical, etc.). Alternatively, FcγRIIIa158 antibody immune complexes may be revealed (and/or quantified) using a second reagent (e.g. antibody), labelled, that binds to the anti-FcγRIIIa158 antibody, for instance.

The above methods are based on the genotyping of FCGR3A158 in a biological sample of the subject. The biological sample may be any sample containing a FCGR3A gene or corresponding polypeptide, particularly blood, bone marrow, lymph node or a fluid, particularly blood or urine, that contains a FCGR3A158 gene or polypeptide. Furthermore, because the FCGR3A 158 gene is generally present within the cells, tissues or fluids mentioned above, the method of this invention usually uses a sample treated to render the gene or polypeptide available for detection or analysis. Treatment may comprise any conventional fixation techniques, cell lysis (mechanical or chemical or physical), or any other conventional method used in immunohistology or biology, for instance.

It will be appreciated that any suitable method can be used to determine the genotype of a subject. Representative methods are shown for example in Dall'Ozzo S, (Andres C, Bardos P, Watier H, Thibault G. Rapid single-step FCGR3A genotyping based on SYBR Green I fluorescence in real-time multiplex allele-specific PCR. J Immunol Methods. 2003; 277:185-192) and in the Examples of International Patent Publication no. WO 03/035904 by Watier et al, both of which references are included herein by reference.

Further aspects and advantages of this invention are disclosed in the following experimental section, which should be regarded as illustrative and not limiting the scope of this application.

### EXAMPLES

### Example 1

### The homozygous FcγRIIIa-158V Genotype is a Risk Factor for Heparin-Induced Thrombocytopenia in Patients with Antibodies to Heparin/Platelet Factor 4 Complexes

### Patients and Methods

### Control Groups and HIT Patients.

The Ab− control group consisted of 86 patients who had undergone heart surgery with cardiopulmonary bypass (CPB). All had received high doses of unfractionated heparin during surgery and tested negative for anti H/PF4 antibodies between the 8th and 10th post-operative days.

The Ab+ control group consisted of 84 patients who had also undergone CPB and had received heparin for at least 8 days. All had developed significant levels of Abs to H/PF4 but without thrombocytopenia or significant fall in platelet count (i.e. greater than 40% compared to the maximum post-operative value).

The patient group (HIT) consisted of 102 individuals with definite heparin-induced thrombocytopenia, including 47 who had undergone CPB. All had developed delayed-onset thrombocytopenia, with thrombotic complications in 41 cases, and both positive H/PF4 ELISA (Asserachrom HPIA*, Diagnostica Stago, France) and serotonin release assay (SRA) had demonstrated the presence of HIT Abs (Pouplard et al, Am J Clin Pathol. 1999; 111:700-706).

Blood samples were collected after obtaining informed consent according to the principles of the declaration of Helsinki.

### FCGR2A and FCGR3A Genotyping

FcγRIIa-131H/R polymorphism was analyzed by an allele-specific restriction enzyme digestion method (Jiang et al, J Immunol Methods. 1996; 19955-59). FCGR3A genotype was determined by a single step multiplex allele-specific real time PCR assay (Dall'Ozzo et al, J Immunol Methods. 2003; 277:185-192).

### Statistical Analysis

The Fischer exact test was used to compare frequencies of FcγRIIIa and FcγRIIIa genotypes between HIT and control groups. The Mann-Whitney U-test was used to evaluate differences in platelet counts between groups of patients. Statistical significance was set at p<0.05.

### Results and Discussion

FcγRIIIa is the only human FcγR recognized to date as having an important role in the pathogenesis of HIT (Reilly et al, Blood. 2001; 98.2442-2447). The frequencies of FCGR2A-131H and —R alleles in the Ab− group, and in patients without HIT but for whom significant levels of anti-H/PF4 Abs were detected (Ab+ group) (FIG. 1, Table 1), were similar to those of healthy Caucasians (van der Pol et al, Immunogenetics. 2003; 55:240-246. Frequencies in the HIT group were 0.535 and 0.465, respectively and there were more homozygous HH patients, suggesting an over-representation of the FCGR2A-131H allele (FIG. 2A). However, the differences were not statistically significant (p=0.16 and 0.39, respectively), in agreement with a recent meta-analysis which found no variation in the distribution of FcγRIIIa-131 genotypes in patients with HIT, with or without thrombosis, and control populations (Trikalinos et al, Blood. 2001; 98:1634-1635).

FcγRIIIa (CD16) is another polymorphic cell receptor for IgG which is also involved in antibody-mediated cytopenias (Clarkson et al, J Exp Med. 1986; 164474-489; and Meyer et al, Blood. 1998; 92:3997-4002). FCGR3A-158V/F genotypes and allele distributions did not statistically differ between patients with antibodies to H-PF4 (HIT and Ab+ groups) and those of the Ab− group (FIG. 1, Table 1). In addition, no difference was found between patients who developed high titers of antibodies to H-PF4 (A₄₉₂>1.5) and those with A₄₉₂ values between 0.5 and 1.5. The FcγRIIIa-158 V/F polymorphism is therefore unlikely to have a role in the immune response leading to the synthesis of heparin-dependent antibodies. In contrast, when comparing HIT and Ab+ groups, both composed of patients with significant levels of anti-H/PF4 Abs, the frequency of FcγRIIIa-158V/V homozygotes was significantly higher in HIT patients (21.5% vs 9.5%, p=0.02). In addition, among the subjects with high titers of antibodies to H/PF4 (A₄₉₂>1.5), the homozygous FcγRIIIa-158V/V genotype was considerably more frequent in patients with HIT compared to those in the Ab+ group (19/88 vs 1/34, p=0.01) (FIG. 2B). In certain clinical conditions such as after heart surgery, a high percentage of patients develop heparin-dependent Abs to H/PF4 (Pouplard et al, Circulation. 1999; 99:2530-2536) However, only a few of them will present HIT. Forty-seven patients with HIT had undergone (CPB and the homozygous FcγRIIIa-158 V genotype was present in 10 of the 46 tested (21.7%) (FIG. 1, Table 1). This clearly suggests a shift of VV homozygotes from the Ab+ group to the HIT group and probably explains the lower frequency of FCGR3A-158V allele in the Ab+ group. HIT is associated with a shortened platelet life span (Wahl et al, JAMA. 1978, 240:2560-2562). Clearance of anti-H/PF4 Abs-sensitized platelets by FcγRIIIa-bearing cells and thrombocytopenia might therefore be enhanced in immunized patients expressing high affinity FcγRIIIa-158V receptors, particularly in those with high levels of HIT antibodies.

Platelet count was also compared to antibody levels and FcγRIIIa-158 V/F polymorphism, and appeared lower when levels of antibodies were high (A₄₉₂>1.5) in VF patients of the Ab+ group (mean=240×10⁹/L; n=18) compared to FF subjects (312×10⁹/L, n=15), but the difference was not significant (p=0.07). High IgG1 and IgG3 binding phenotypes in healthy donors were associated with at least one V allele (Wu et al, J Clin Invest. 1997; 100:1059-1070), but we did not find any over-representation of VF heterozygotes in HIT patients. Similar results were found in autoimmune thrombocytopenia (Carcao et al, Br J Haematol. 2003; 120:135-141) and in lymphoma patients for whom a good response to rituximab was only associated with the homozygous VV genotype (Canron et al, Blood. 2002; 99:754-758; Weng and Levy, J Clin Oncol. 2003; 21:3940-3947).

In this study, FcγRIIIa-158V homozygotes also appeared more frequent in HIT patients with isolated thrombocytopenia (24.6%) than in those with associated thrombosis (17%) but this difference is not significant (p=0.36). If confirmed in larger populations of patients, this trend could support the fact that FcγRIIIa receptors are more highly involved in the pathogenesis of isolated thrombocytopenia. Alternatively, the role of platelet activation and consumption could be greater in cases of thrombotic complications, but this hypothesis warrants further study.

In conclusion, our findings strongly support the hypothesis that homozygous FcγRIIIa-158V/V patients are at risk of HIT, particularly when high levels of antibodies against H/PF4 complexes are present. They also indicate that Fc-receptor-mediated reticulo-endothelial clearance of platelets is a putative mechanism of thrombocytopenia in HIT, and this could in part explain the efficacy of high doses of intravenous immunoglobulins reported in some patients. Frame et al, Ann Intern Med. 1989; 111:946-947; and Grau et al, Am J Hematol. 1992; 39:312-313)

The rule of FcγRIIIa in the clearance of sensitized blood cells has previously been demonstrated in monkeys (Clarkson et al, J Exp Med. 1986; 164:474-489) by the use of a blocking anti-FcγRIIIa antibody which reduces the clearance of sensitized erythrocytes and improves platelet counts in patients with immune thrombocytopenia (Clarkson et al, N Engl J Med. 1986; 314:1236-1239). Further experiments are thus necessary to determine the respective roles of FcγRIIIa and FcγRIIIa in the development of HIT in humans.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method of assessing the susceptibility of a subject to drug-induced thrombocytopenia or selecting patients for an a treatment known to induce or suspected of being capable of inducing anti-platelet antibodies, comprising determining in vitro the FCGR3A158 genotype of said subject.

2. (canceled)

3. A method of improving the efficacy or treatment condition or protocol of an antithrombotic treatment in a subject, comprising a) determining in vitro the FCGR3A158 genotype of said subject, and b) adjusting the treatment, optionally adjusting the selection of composition to be administered, adjusting the dose or administration schedule of a composition for the subject so as to obtain a better clinical response or reduced risk or degree of thrombocytopenia.

4. The method of claim 1, comprising determining an amino acid residue at position 158 of FcγRIIIa receptor, wherein the determination that a subject has for a valine at position 158 is indicative of an increased susceptibility to drug-induced thrombocytopenia, and the determination that the subject has a phenylalanine at position 158 is indicative of a decreased susceptibility to drug-induced thrombocytopenia.

5. The method of claim 1, wherein determining the amino acid residue at position 158 of FcγRIIIa receptor comprises a step of sequencing the FcγRIIIa receptor gene or RNA or a portion thereof comprising the nucleotides encoding amino acid residue 158.

6. The method of claim 4, wherein determining the amino acid residue at position 158 of FcγRIIIa receptor comprises a step of amplifying the FcγRIIIa receptor gene or RNA or a portion thereof comprising the nucleotides encoding amino acid residue 158.

7. The method of claim 6, wherein amplification is performed by polymerase chain reaction (PCR), such as PCR, RT-PCR and nested PCR.

8. The method of claim 4, wherein determining the amino acid residue at position 158 of FcγRIIIa receptor comprises a step of allele-specific restriction enzyme digestion.

9. The method of claim 4, wherein determining the amino acid residue at position 158 of FcγRIIIa receptor comprises a step of hybridization of the FcγRIIIa receptor gene or RNA or a portion thereof comprising the nucleotides encoding amino acid residue 158, with a nucleic acid probe specific for the genotype Valine or Phenylalanine.

10. The method of claim 4, wherein determining the amino acid residue at position 158 of FcγRIIIa receptor comprises:
Obtaining genomic DNA from a biological sample,
Amplifying the FcγRIIIa receptor gene or a portion thereof comprising the nucleotides encoding amino acid residue 158, and
determining amino acid residues at position 158 of said FcγRIIIa receptor gene.

11. The method claim 4, wherein determining the amino acid residue at position 158 of FcγRIIIa receptor comprises:
Obtaining genomic DNA from a biological sample,
Amplifying the FcγRIIIa receptor gene or a portion thereof comprising the nucleotides encoding amino acid residue 158,
Introducing an allele-specific restriction site,
Digesting the nucleic acids with the enzyme specific for said restriccion site and,
Analysing the digestion products. i.e., by electrophoresis, the presence of digestion products being indicative of the presence of the allele,

12. The method of claim 4, wherein determining the amino acid residue at position 158 of FcγRIIIa receptor comprises: total (or messenger) RNA extraction from cell or biological sample or biological fluid in vitro or ex vivo, optionally cDNA synthesis, (PCR) amplification with specific FCGRIIIa oligonucleotide primers, and analysis of PCR products.

13. The method of claim 4, wherein determining the amino acid residue at position 158 of FcγRIIIa receptor comprises a step of sequencing the FcγRIIIa receptor polypeptide or a portion thereof comprising amino acid residue 158.

14. The method of claim 1, wherein the subject is a human subject.

15-19. (canceled)

20. The method of claim 4, wherein said drug is an antithrombotic drug.

21. The method of claim 4, wherein said drug is heparin.

22. The method of claim 4, wherein said drug is a GPIIb/IIIa inhibitor.

23. A method of assessing the susceptibility of a subject to heparin-induced thrombocytopenia, comprising (a) determining whether the subject has antibodies to heparin/platelet factor 4 complexes and (b) determining in vitro the FCGR3A158 genotype of said subject.

24. The method of claim 23, wherein step (b) is carried out if a determination is made that the subject has antibodies to heparin/platelet factor 4 complexes.

25. The method of claim 23, comprising determining amino acid residue at position 158 of FcγRIIIa receptor, wherein the determination that a subject has for a valine at position 158 is indicative of an increased susceptibility to drug induced thrombocytopenia, and the determination that the subject has a phenylalanine at position 158 is indicative of a decreased susceptibility to drug-induced thrombocytopenia.


SEQUENCE LISTING (11 sequences)

SEQ ID NO: 1 - DNA, 21 bp, Artificial Sequence
  Description of Artificial Sequence FCGR3A specific primer.
atatttacagaatggcacagg

SEQ ID NO: 2 - DNA, 20 bp, Artificial Sequence
  Description of Artificial Sequence FCGR3A specific primer.
gacttggtacccaggttgaa

SEQ ID NO: 3 - DNA, 32 bp, Artificial Sequence
  Description of Artificial Sequence Amplification primer.
atcagattcgatcctacttctgcagggggcat

SEQ ID NO: 4 - DNA, 32 bp, Artificial Sequence
  Description of Artificial Sequence Amplification primer.
acgtgctgagcttgagtgatggtgatgttcac

SEQ ID NO: 5 - DNA, 22 bp, Artificial Sequence
  Description of Artificial Sequence Amplification sense primer.
ggaaaatcccagaaattctcgc

SEQ ID NO: 6 - DNA, 27 bp, Artificial Sequence
  Description of Artificial Sequence Amplification antisense primer.
caacagcctgactacctattacgcggg

SEQ ID NO: 7 - protein, 254 aa, Homo sapiens
  misc_feature: Amino acid sequence of human FCGR3A158F.
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 8 - DNA, 22685 bp, Homo sapiens
  misc_feature: Nucleic acid sequence of human FCGR3A158F.
cagcctggctgacacagtgagacctcatctctaaaaaaaaaagcaagcagaatattttcttaaaaggcaattatattccttcttggccaggcccagtggctcacacctgtaatcccagcactttgggaggccgagatgggtggatcacctgaggtcaggagttcgagaccagcctggccaacatggcgaaaacccgtgtctactaaaaatacaaaaattagctgggcatgggggcatatgcctgtaatcccagctacttgggaggctgagacaggagaatcgcttgtacccgggaggcagagattgcagtgagccgagatcatgccactgcactccagccttggcgacagagtgaggctttgtctaaaaaaaaaaaggtattttttgcctctctgttggtaccaattgttaaattctttgtggaccactgatgcttaccaaaaaaaaaaaaaaaaaaagtgggggcatcatatttcctctagttgacattaagacacagtaatttagccagaggagatcttagcaaacatacagtccacactccactttctcatttcatgattgtagagactgagatctagacaatttaatcggtggtcaccctgggtgacatagctaggtctagagctcctggtctccaggtcagcatttctttcttcttcattaaatgtcaagtttcctcccctgttcattattagctccttccagaaagagagtttcttatctttttagtaggtactcagtaaataccaaggtattcactaggatgcctttggatgaaggtaacaagccctgacttaaattggcttaaacagcagggaaatttacttgaaattgtaagaaatctgggttggttgcgttagaagctcagtgatgtcaccaagaccatattctatccctccactctgtcctccttggctatttggcattgacctcagactggctgccttcaaaatcttaggttttgccagcagaacctaggacaaaatgagcccttgttcatgtacagtgggagagagagatcatctctcccaaacatggcactcccctctaccagattggccctatttaggacaaagttccgtcttctcccacttaaccaataaaagccaggggaatgctacaccctgagtggcttagatcagtcaagatccacctctgcatatgagggtgattcctgaatagaatcaaggttatattagaagggagggagagggatggatatcaggctagtacatcatattctatttgttgagttaactgagtcatagcaattgttgagttggaaaaaactcagaacctactgtggattcaagttcaagaaatcattctttcctacatacaacagcattgctctgtagccctgagctaagagagcatcacgaaatacagtcttcttgctgtttataatcgtaagcaaactcttggacctgggaggggatgaatggataatgtctgtctgacttgcttctttctagttagtaccaactacctcccttcttcctgtgattgttcttagaataggataaaaaatcttcccttccctagatcttacagtctccccttcccccaggcctttctatttttcaggattttactctaatcacaccaccgaagaatcaagaaatctttaaagtgtattagagtagctagttgtggcagcactaaaacacggctgcaaattctttgacactctctccatcaagaaatgaggcctacatcctctacccttgaatctgggtgggcttataacttctggtgattagactacagcagaaagagaagctgtatagcttcctaaatgttataaagtcttaaggatagctgccagcaagaccttaggacaaaatgagcccttgttcacgtccagtgggagagagagagagagaccctctctcccaaatatggcaatcctctctacctataacatttacataaatgttataggttaaacgttccacaacaaactaagtactatttaacatcaagaggaaaaagagacaggagaaagggttaataagcctgttgatgaggatctaagaagaacaaaggaggcctggtttgggcctggctacccgttggtcttgcaaagaagagtctgaggtggcagagccttcagtggcagatgccaaattatcatcatgagtgactgcaagacagtgtcagctaagatagccatttcaagctgctgaaggccttctcttttagtcgtggagtcctgtgataagaactgaaagttggaagagtgtgcttgtctgtggccttatttggtcggatgcagtctttatcatttttaatttgtttcttagaacattttatcttgttggccaaatgccctacgaaatataaaatggagtctttttctaagatggagttagttatgtcaagggtcctttatacagtcttcatcctttttcctggcatacaactcctaaaatccttagaatctccaaagtgatgtcttttggtgtgctaatgaggtaactgatggctggcagctcttaggtagcttcataacaggggctgggcacaagaaagatcatggcaaggtcagaggattggggctttcagctccaccctccaaactccctctgggaagtggagaggggctgaaggttgaattgatcaccaatagccaatgacttaattaatcattcctaagtaataaagctcccataaaaacccaaaaggacagggtttggagatcctccagagagccgaacacagagaggttcttggagggtagtgcaccagagggcatggaagctccaagccccttcccacaggtcttgccctatgtactctttacttgtgtcctttgtaatattctttatcacaaactgataaatgtaaatgtttccctgagtactgagagccactctagcaaattaattgaacccaagatgcaggtggtgggaacccccatttataactggttggtcaaaagcacaggtaaaacaacctggggcttcatcctggagtatcagaagtgtcttgtgagactgagcccttcacttgtgtcacctgatgctatttccagttagatagtgttggaattcaattgaatttgagcagaagtcccaatccccagacctgtagttgtcagtgacctcttaggaactgggctgcacagcaggaagtgaggggcaggtggggagcaaagctttatctgtatttacagtagatccccatggctcacatcaccgcctgagctcctcctcctatcagatcagctgtggcattaaattatcacaggagcatgaaccctattgtgaagtacgcatgcaagggatctaggttgcattctccttatgagaatctaatgcctgatgacctggcattgtctcccatcaccctagatgggactgtctagttgcaagaaaacaagctcggggctccccctgattctacattatggtaagttgtataattacttcattaaatattacactgtaataataatagaaatgaagtgcacaataaatgtaatgcacttgaatcatcctgaaaccatcccccacccccctacccctgtccgaggaaaaattgtcttccatgaagccaatccctggtgccaaaaattttggagaccactggattagaagacacccagttggtgcccactgctgaattgcttgcttgcttgcttgcttgcttgccagtggagagaaatccccacatatctgttgtcagaaatgtgttgtgagagcatagtgggaggaactgagtttgttttttctacagttacagcaataggtaactggaattcaactgctggactataccaaagactgccaggccagcctacctttctcacagccttcttgactacctgtcttggatgagctcactgaaagcccacataccttcattctagcatttcctcagtctggttgagctgctttggaggtaatacaggttgtaggacttccctccactcctgctcaggaccgttttcagcaggctaatcagacagcagttggcactgagtacaactggagaaatgttatcagcactgaagactgctccagctacaaatgcacagcgacccacctcagctggacctttggcattgcttggcaatccttacaactgtttggaattcttggtaattcttgtacatacaaacagtcggctccttctcccatgcaccacaatggccattctcaactctggtgttctcaaacccccaccacaactgttacccactctttctcgacaagtgtctttgactcctccttaaccaagaaaatcaggaccagcagatgtggatggtcactcaacatctgaaaatggatttgcatatgtaccccctcagctcctgccttcagctcagagcgagaggtaatccgtatccagttcacagccaactccctgtccatgtcccattcccatctcctcaggacccacacttgcttctctaggtgttccgtccctgttaggcatccaacttctcccaccctgctggcttcttcccaaagacctataaccaagctcgtatctttcacttaaaagaaaacaaaaactcccattctcttgtaactacctttgcagaattgtgtctgaaacagtgagagagatctaacttaattgactccatcttgcttctaacctccaagctgtctttcctcattcttgggcataggctgaactaactttgggagaaacttagtttatagtttgtggtttaaagcaaagatgataacagccctttcccagggcagacctcctttttttctgaagactagattgtctttgtaggactaacattagccacaagattggaaattatggtttaggaatcatgcaggtggaggctacaagattctgacccttcctaagcactgatcctaagatcggtgcttgagatattttgcagaccctgcacttgatggatcacctggcaacacccagatcaataaactggctcatctgatcttgtggtgcccacccaggaactgactcagaacaagaagacagcttcaacttcctgtgatttcatccctgaccaatcaacactcctggctcactggcttcccctccaccaaccaagttgtccttaaaaactctgctccccgaatgctctggaagactgatttgagtaataataaaactccagtctctggctcagtcagctctgcatgaattactctttctctattgcaattcccctgtcttgatgaatcagctctgtctagttaccatcctccacttctcctttcttattgtgtcacttagggctctgggttataaacaactttatcagaatccagatcttttaagtagaggaaaaagatttattggatggaaactagaggaggtgagcccatcccactgctgtgatgactgggacccaaccatctccctctcctagaagtgaatctcccttatgagtaaacaagtgtcacttattcaggattcatctcagaagagactctaatgggccaaacctcagttatatgcctgtcctctgtctgcctgtatcagttagctagcacttttataacaaagtaccacagactgggtggcttaaacaacaaaaatgtattttcttacagttcttgaggctgcaagtccaagatcaaggtgttgacagggttggtttcttttaattttttttttaaaaattttattttagatttaggggtatgcgtgcaggtttgctacataggtaaactcctatcacgggggtttgttgcacacattatttcatcacccatgtactaagcctagttactcaatagttattttttccgatcctctccctcttcctaccctccaccctcaagtaggccccagtgtgtctgttgttcttcctttgagtccatagggttggtttcttctgaggcctctctccttggcttgtaggtgtccatcttctccctgtatgggtctgtgtcccaactaacaaggacatcagtcatattggactagagcccacctaatgattttattttaacttaattacctctttaaaggctctatatccacatatagtcacatcctgaggtatagggggttccacatgtggacttcaacatatgaactgggagagacaaaatttagtccttaatagtgccccaaagtggggaaaaggaagatctggaccctcgggtttccatagtagaaagcaatcactgctttctattaagtactcacagtggggcttctccagaaagaatgatatgctaataagaaggggaggaggaagtgatcctggacagccagatgatatgtgcactattccttcataatggagattctgaagaggagaagcacttgactaaacactttttcattcctactccgtcttcaaccaaaagctgtcaaacttctgtttctcagccccagcccctgaaattgctcaggaaaaggtcattaatagttccttgattgccatatttcaatcaaactcttgtttgaattcttttctacaacattaatactgttactgttgactactccttccttgaagatctgttcccactaaacttccttgttccctcctcttcagcccctcctatacaaactcctttgtcagctatttttcctgtgcacgcttcaaaaatgtttgcatgtcaagtttctgtcattgactctctcctcttctcctctccctctcaatccctccttcctttccctcactgtttccctttaattctctctcaatactcttacagtttcagagatcttatccttactttatcttaacctaggatctctggatggattcaaatagagcttcttaaattaaaggaaacataatgtgtatatttgcatcctttcttgggagaaggcccaaaggtttttatcagaggtttgaaacctcaaccgtgttggtgcctcctaaattgtgtcttttgtcaagacctgtcttctgagttccagggccatgtgtctcactgcctactggaaatcttcacctgaaaccttcacagctacctcaaactcaataacatcaaaagctgaaatcattgtctctccctcccaaagcctgctcatcttcccatttttcttttgtccatgaaagctactgccatcctccttatcacccaaattagaaatccgagcatcacccagacctctcccccttcatcacccctcagccaatcactcaccaagtcttgtccatccttccttcctaacttctctcctggatgcttccattgcatatccactttttaaacagagtggctcttgtctcaactagactgttgaaataatcttccaacttttccctccaccttccatctctctcccctctaactcattccttggactgctgtcagagtacttttcataaaatataaaacagatcttgtgattccccagtctaaagcctttttattagttcccattaccttttagaataaaatatgtactgttcatcctgacacacaaaactcttcgtgataaatactaattgagtgcctagtatgtgcctgcccttgtgctaaatgttgagggtacaggggtaaacaaggtgaacagcttccctgctctccaagacctttcagtccacaaatgcaatgagtttacagaggagaagcacaagctcctaaaggagttggggtggggttgggggtcagaacctaatttagaaaattgaggaaggtctcaacctcccatcttgcatttacaatagtaatcagcaggtgtggtaccaaatatggaaccaacaattttatctgcattatctcatttaagccatgagtgccattattgttagcctcactttacagataaggaaactgagggctagaaggttaaataagtggcagagttgggatttcctccagattcctgtgagacccagacatcttaatccttttggaacctgtgcttctcctttgtagtactcactacacttgtggaactacatccaactacacttgtggaactacagccagctctgcaaacatgacagtctacttcactccaagtctttgctcatgctgctcctcttgcctggaatgcctatttctctcaaaaatcttcctgctgaatattttgcgatctaattaaagtgttctctcttccatgtacactcctccctcagatagaattagccactgtcttctttgtgcatacacagcatttcataaatactgtcacagtccctctagcacttcaaatacttatctgatgttctccccctaagaaactgtaagtcctagaggatgacaatcaactgaattccatagtcagaaacttctgctgtgcctggccttccaatgagaaaaggagagaagaggaggggaaggaagaaaaagggaaggagaagaaagaaaagcaaacatgaagataaacacttcaatatatgatatcccaagaccatctacccttttgtaaaaattttgctttttttttttcccccccaagagtcagggtctcactctgtcgcccaggctagagtgcagtgccatgaacataactcactgtagcctctaactccggggctcaagcaatcctcctgcctcagcctcctgggtagctgggactacaggcatgcaccaccacatctggctattattattattactatattagtagagatggggtctttctatgttgcctaggctggtctcaaattcctggcctcaagcaattcttccacctcacattggccttccaaagtgctgggattacaataagccaccataggccaaaattttgcattttatccattactgtaaaattaacccttagaaatccaacaacactcaatttgagaattgttcaacaaccacttaatgaaaaccccctgaaagcttcccatcctgttgcagtccctttctctcctcctgtgctctctcctcttcttcctatctagcccacccttttggcagctaagaattcctccctccattggagagccacagaccaaagaggagtcaaataagaaaataagacctcaaagaaggaaaacaaagtgaaggccttgcatcagaagtcacgtggcagaaagccacctggatatctgaaaagaagaaagaattgagggatatccgctttttgcctcagagaccatccttagccctgaaggctttgtttctgctttaggtttcccagataagcatccgaagtgctacagcaaggaactttaagtttccagatacttgtctggattttgcaaggcgtagatgagtcacttgagaaggagaactggaatggctgcctgggttcatttccattgtccaatccaagggcctgtggagaaggggctgctgcaagactctgtgtgtggcggggggaggggtgggtacgtggatggcaatgggaggatcaattaactccacccaggagccaaatgaaacacacaaataaaaaacaaaacctgagtagtggtttttaggtcattctggagtagaaagagcattcatttatagcaaaggttggcgggcacctgtgtcagcccctgcctccactccacccctaacaagtatcaggtgcccacacgggcctgctgctcgcctcctgggcttttctaagccaggtgagacctgtcccagatgtccacgaatccactgggggagtggcactatcaagcagagtcatctgattttctgcctgggacctggaccattgtgagagtaaccaacgtggggttacgggggagaatctggagagaagagaagaggttaacaaccctcccacttcctggccacccccctccaccttttctggtaaggagccctggagccccggctcctaggctgacagaccagcccagatccagtggcccggaggggcctgagctaaatccgcaggacctgggtaacacgaggaaggtaaagagttcctgtcctcgcccctccccacccccaccttttctgtgatcttttcagcctttcgctggtgacttgttcttccagggcccatttctctaccctacctgggtttcttctaacctggaaatctaatgatcaaatcacactaaaaagtcagtagctcctgtggattacatatcccaggagcatatagattttgaattttgaattttgaaagaaattctgcgtggagataatattgaggcagagacactgctagtggtctgaagatttgaaaggaccactttctgtgtgcaggcagggcctcagctggagatagatgggtctgggcgaggcaggagagtgacaagttctgaggtgaaatgaaggaagccctcagagaatgctcctcccaccttgaatctcatccccagggtctcactgtcccattcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcaggggctggggaaaggctgtttacttcctcctgtctagtcggtttggtccctttagggctccggatatctttggtgacttgtccactccagtgtggcatcatgtggcagctgctcctcccaactgctctgctacttctaggtaagtcagggtctccctggttgagggagaagtttgagatgccttgggttcagcagagaccccttttcaggctacgaatgagactcccacgaagggatgggacccctcaccacatctatagctgtggattgagctcctaggacaagccaagatggggctagaaatgaggagaatgctggttccaattggggcatactcatgagtgaggccagtcacttcacccctctgggtcccagaatcactctgtggaaccaaagagcttcgactagatggtccctagggtctgtctctttcagtttgacattccagggttctcctctatgattttcaatttctaccctttcttgtggggatatgggttgaggctctttctgtagcttggttcagggaaattcaacctgtacccttaatttgtgagtttgcacagggagcaaggggtaagggagcagtgttgaaaatagggatttgtgttgacagtggcgcaagaggcatgaacagtggagaccagagagcaggtagcaaggtttccaccagaaacatcctgattcttgggaaaattgggctcctggggcagaggagggcaggggagttttaaactcactctatgttctaatcactctgatctctgcccctactcaatatttgatttatcttttttcttgcagtttcagctggcatgcggactggtgagtcagcttcatggtcttggattgacccagtggggcacatatggggacaaaggccataagatattgggaaatgcttgttgaatgggaaaatgctgatgtggggttagcagggatagttcctccaacacagcagaacttggccctgtgcttctctggccagctttccttaagatactgaacaggccaaaaatggggccaagatgctctaagactgagccaccaagcatgggtttgcaatgagctcattctggctttgaggctccctgggaatggcagtgtagagcctgctcctctccctgtcctcaccccacattatcttggctcctcagaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggtgagttgatgaaggggaagaggaaaatcaccaataaagggtgaaacaaagggtcctgaaatacttggtaagagccagagatgatattcttagagataaaagctaagatgagatgatgtgtggtcccactgaatggtatcagagttgtagtcctagctctaagtaggtcttgggcaaaatgtcaaagcctgtcagacagtagatataggactgctgcattgcacaattccaagaatccccatatggagtgcatacaatgtgaatgtgtcatgtgaaggttaggccatggcatagatgctcaataatagttatttatatatttattttcattttttttaattttattttttgagacagagtatcactctgtcacccaggctggagtgcaatgcggcaatctcagctcactgcaacttctgcccccttgggttgtagtgattctcctgcctcagcctcccgagtagctgagattacaggcacccgccaccacgcccagctaatttttgtatttttagtagagacagggtttcaccatgttggtcagtctggtctcaaactcctgacctcaggtgattcaccagccttggcttcccaaagtgctgggactacaggcgtgagccaccacacctggccaataatatttattgaataaattaatgaatttggtgttaggacctcaatctccttctcgctctcagacatgtaatgccctaagccacctcccaaagcaatcctagtggcctagcatcatatctttctgtctcctcatcaatgctatactcaaacctataattaagcataaatttggtaatgtgatagctcttccaatagaggcagatacatgttcagcctgcacattaatcatgacatgaaagttcttgtgtactattaacagaatatagacgtcagacacaggtaggagaaatattttgaaggcagaggtctttcctggtgtccctacaatcttaccacataggctggtccctgcagtgtcgccctgcaaacctaactctacttccacggctgttccattcatacaatgtttatgggtggaacaagctttgggggaagaagggcataaggaggtggatctgcaagagagctccatggaattgggcctctgaaactgatttttgtggctctttggcctctgacagtaccactcaactgacatggtcttcactctccagagctacaagaagatatgtccatttctagctaggtaagagatgtccacctacaaccaaataaaatgggggaattaccaagagaaagcaatagaaaaatcaagtctaagagttactagtttgccttgaacttggctctagaaactggctttagaagtctagccaatcaaggctatattaaactgtgaccatgagaattagcttcaccaggtaaacttctgagcatcctttaatcctttaggacccatttcacttatgtcctcctctgagaagcattttttacttctttttttgtttgtttgtttgtgtttgtttttgtttttgtttttgagacagagtctctctctgtcacccaagctggagtgcagtggcgcaatcttggctcactgcaacctccacctcccgggttcaagcaattctcctgcctcagcctcccaagtagctgggactacaggtgcatgccaccacgcccggctaattttttgtatttttagtagagacagggtttcgcagcgttagccaggatggtcttgatctcctgacttcatgatctgcccacctcggcctcccaaagtactaggattacagatgtgagccaccgcgcccagcctgcattttttacttctttcaggcagaatttctttattccaatctagtcagccccgcagtcctttattcttagcctgttgtagcacttgtcatattgtattgtgattatttctgaatatttatgtttctatgtctagactgtagattctttgaggctgagaactatatgtcccatcatctgggtatctccagtccacagtgtgtcatacatagtgagtgcttgatgaaatatcacttgaaggaatatacatatggacattcactgggtccatgacaggatagattcgaacaagaatgttcctccaaaggccaccagactatatactaaccatgactttatgctaataatgattcatctctctgctgaaaaagtaagtggatagataggcacatggcttcttttgataaatgatatctcttaataggtaatgaagattactttctgtttggcaaatctttgtggtagagaatcatgaccaacacacgtcctaccaattttgtttagcatcaggtagtagattttttaaattatagtaattcaagctgagaatgtagatttaaaaaataaaattattgtaaattttgttttgttcttattacaaaagtcatttggggtcaatttcaaaaatatataaaagtaaacaggagaaatttaaaatgtccttcagtcccactccttcagagaaaacccctgttaatatgtaagtgcatatccttcttttttctgtgcataatactttttaaaatatttgaagtattatgcttttttaacttaaaattgtctcatgaatattttcttatgccattataatacttacctataacatcattattttttaattattcaggccctttcccgaccatgacctcatgttctctctttgtgaagtctgattacttggtgacatgatcgtgagaataagctctggcgatataagaatttcctctcttgaaggccatgctcagtaaattacttggtgacatgatcgtgagaataagctctggcgatacaagaatttcctctcttgaaggccatgctcagtaataaagttggtctcaccgaggccctgtgacaccttagaaaccacgaattgccaggctgagcaataccagtcccgcccttcccctccctggtgtttacattgagttctccttcacaatttctgcagccactccgtggccaccgtcaccttattcctgactgccacaagagtctttcaatattcctttgattgcctattccttctgaaatctaccttttcctctaatagggcaattcatcattttcaaatgcaatttttactctgatctagaacttactgtgaatccttgtcacctgccacagcaaatctaagtctagcacttaaggatcctgcagatatgctcatcgttgcttctcacttacctcattgcttagtccctctgctctaaccctgtgtgttgatcacatgtgtgtgtgtccctcttccccattagacaaaggtcttggtatgacttcagttctcttgcagggccccatcagctcttccccaaagggagctatgcagggttgactcccaatctggctttcccttatgtctcaggatctgggtggtacgtggccccttcacaaagctctgcactgagagctgaggcctcccgggcctggggtgtctgtgtctttcaggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtgagacatgtgccaccctggaatgcccagggacgcctgtgtgtggaacctgcaatcacactgggaagttgagttgggaggagattcctgattcttacacgcacttcttcatatgtggttccctcctggtgatcaccaggaggtccccaaaagtccctgattgcagggtaggtttgcagctctgtttcagtccattcttttggggtagctaggaggtgtcattcactctgcagcatgatggcaggagcagaagccacatctcctccccaataaatacctctgtctttccttacgctaatcacacccacggtgtcatatgttcctatcgtgctggcctccttcttatccaagccttttagccacgatccaaactggcaggagcccctcatcccctcacagaaagagcccagaacctgggttctggccctgcagctaattaaccatctgaccagaggtgagccacttagtctctctgaaccccaatttcttcttccgtaacaaaaataagctgacatttattgggcacctttcagtgtgctagactctgtgctaaacaattctttacatgcacctggtttgactatcacagtagaccttcacaacatgagataggtaatattccattttacagatgaagtaaccgaggtgcaaaaataaataaataagtttccctaaggtcacatcaaagacttcaaagcctgtatatttaaccagtaagtaaaagatttgaacaagcactaatatcctatgatcccattaagtcatccacaaaacatctctaggttctgtagcaccagcctccagaatcagagctctagagtggtgtgcctggactttccagtttcacagaacttctatctgtaactagcccaagacataaattgtaaacaatttgcatgtagaaaggcagcaaaacaccttttgagattttgacactacaatgccataatttgtacaaaaataatttcatgacactttaaactgaaagtaaatactcccaagtggttagggaaagagagcaaataaagcaaatggggtaacatgtaaacaatgagtggatctgggtaaaggatatacgagattaaactattctggtcattttttttttaagtttggaaatatatcaaaatcaagagtttaaaaaattgaaatgcaaaatcaacaaatttgtcccagtttctagaccatagcattgtctgacaatttcttaactgtcacacaaaacccagcttacaacctaacttgttaacgctccctgtcacatctctgtcaaacaagcaggagcctttgctcagtgtttggtgagctgtcctctgctcagatagcactaagatcaggaaccaatgggaggaagcaatactttcccccagacttccccaccattcctaccacttgcctgttggctgttgtcaaagactttctactggtgacctcactgtttgttccaaatatctgccttagtgactgtcattttttttcatctctccacttctcctaataggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaattctttcttcttccctttgtccaacattgtaacagagattggtttggattggtaagaaacaccccctcctcccagcaaccatctcaccacaactcatataaattagccagcttgctttccaaatcttgctgagacaattgggctaaggaggattcttatgggaagtatgggataggagggtgaataagcattagagatcgttttagagcattggggcagataggagaaggcacagctacacaggaggtagaggcctgggcagaggtagagggtcagcctgattgtatgaattatgagctatataccaagacgattcaagctagattgcatacataaatattacataagattccgacacgacacaggtgcatttggaaaccttggacattcaactcacatttatttactacctacaatgtgcaagcttgagttcaggtgctgaagataccagatgaacaacacagggtcattccctggagaagctttatttctagtgagaaaaacagttaaataggaagagaatgaagaaagggctgcagaaaagaggcttgatttggggggtgtggtcatgaaggatgagtaggagttcgccaggcaaagaagagaagaaaagcccaaggttcataggcaaagattcaaaaaccagagtgtgagttcaagaaagcagtttggttctgtgtcggtgagggagaggaaagagtttcagggccagatcatgaagggcattaccttccaaactaaggagatcgtatcagaccctgcaatacattgagagagtttaagcagaccaggtttgtaccgtatagtattttagaaggattctctcgcaactacttgatggatggacgggacaggagagttgaagaccagaagccaaatagggcagcaaggcaggatgcagtaacccaaagggagcaatgaggaagtaactggcggtgaggctggagaggaaggtgcttaatcaacaaggtatttaggaggccgactctccaagaattggcagccagcagtacacggcgtgactaaggaccaggttccacacatagtgcccgttttctgagttaggaaatagaaaggcaaggcaggtacaggtttggtggaaagacaaacaattcgttttggtattattagtacttacttcctttggtcagtaaattttcttaaagtgtcagtttccataacgtaattgccgtggttaagcagctaagagttatcactacaaccctagtcggaaaaaccaaatacctcaaaattacccgtacagcactaaggcagaagaggacattgggaaccacacaacgcggaggtctgctaccagagctccctgcggttagcaccgcggctggttttgagcgccaaggccccagcgctcccagcggatagcatcgcacgcagttttttcagtcaaagtttcaaaaacccagggttcacaaaatgcgacttccgtccctgggtgggatcgaaccaccaacctttcggttaacagccgaacgcgctaaccgattgcgccacagagacgggcgttggcgattttggctgccaagtcacttcactgaagaaaaaatgctcagcactcacgtctccaaaaaaattgaggttgatttgaaaccagtgacacaattagctttccgtgcttcagggcgcggctcatagccctgagcgaggcaggtcttttttctgcgctagcacttgcctagatctggagcaggactcagcttccagcagaagaggttgagaaaaggagagcagaagagaatgcaggaacgaagggtcttcggggaatccaaaatggatgctctctgtgggttcgggggttccgttgattttggtcagagaagtacgacgataagctttttttgctgatgtagacaacttatgtatgcatgtgcacacgtttagtgctgactcataataagcttattatcgtgagcattaaaaatattttctttcaggtccaatcacgtccagcaaaatgtgatgtctaagtaagtgagttttgtgttacaaaattagtcttcaacccacgctgttttgaaaggtttctaccggcatattagacatgcagacagaacacggagcttaaaaagcctgtaacattccaattaatggtattcagcttggaaataaaaaatattttttaaaaaatgcgtgcaacttaaggactttcatgctgacatatccagatccaaatatctgaggacagagacccctaattccaccaccatcgacctagggaacctcgtcagtgctgggtctaaaaaggctttttttttttctttaattcatatgtatatatactttattcatatatatatatactttaagttctaggctacatgtgcacaaggtgcaggttcgttacatatacatgtgccatgttggtgtgctgcacccattaactcgtcatctacattaggtgtttctcctaatgttatccctccctccttcccccacccacgacaggtcccggtgtgtgatgttccctaccatgcacacgtatgtttattgtggaactattcacaatagcaaagacttggaaccaacccaaatgtccatcaatgatagactggattaagaaaatgtggcacatatacactatggaatactatgcagccataaaaaaggatgagttcatgtcctttgtagggacatagatgaagctggaaaccatcagtctgaacaaactatcacaaggacagaaaatcagacaccgcatgttctcactcataggtgggaattgaacaataagaacacttggacgaaagccattttctatattgcccaaaaaccagggtctctccatagcctccacacagaatctcctttctttctgccctgccatcctctgtcatcagtgggctccagtttaggagcaggtggaagttttcaatgatgttcagtgaaatgagaagacatgcaaacatagatatgtatatgcagaaattatatatgcatatatgtttatatgtacacagtatcatatgtataataaataagtaaataaataaataaatttgccaaatgatctttaaactagagtcatttattttttttattaatttttttttttgagatggagtcttgctctgtcgcccaggctggagtgcagtggcgcaatcttggctcactgcaacctccacctcccacattcaagcaattctcctgtctcagcctcctgagtagctgagattacagtcatgggccaccatgcccggctaatttttgtatttttttttttttttttttgagacagagtctcgctgtcgcccaggttggagtgcagtggcgcgatctcggctcactgcaggctccgccccccgggattcacgccattctcctgcctcagcctcctgagtagctgggactacaggcacctgccacctcgcccggctaatttttttgtatttttggtagagatgggctttcgctatgttggccaggctggtctcaaactcctgacctcaggtgatcctcctgcctcagcctcccaaactgctgggattacaggtgagagccactgtaccaggcctagagtcatttcttttatactttaaatttttgtctctgttcttttgctcagacctgtggagctggcaatatgggcaagtgtcatggactgtctactgccaggaagctccattgtcaccgacaggatcagaagtggcatggtaaatggtacaagaaagcccattcgggcacagtcctgaagaccagcctttttggaggtgcttctcatgcaaagggaattgggctggaaaaagtagggattggagccaaatagcccagctctgccactgagaagtgtgccagggccaagctgatcatccagcataagctagatgctgtggtctccactggcacagctgatcctcttgttacaggatggaggctgtgaggcagatgagagaacagcaagaaaatcacagcctttgtacctgatgatgattgcttgaattttattgaaaaaaatgatgaagttctgtatcaggggaaccagcacccaatatttcaatgtaggttct

SEQ ID NO: 9 - DNA, 45 bp, Artificial sequence
  FCGR3A gene 158F allele
tcctacttctgcagggggctttttgggagtaaaaatgtgtcttca

SEQ ID NO: 10 - DNA, 45 bp, Artificial sequence
  FCGR3A gene 158V allele
tcctacttctgcagggggcttgttgggagtaaaaatgtgtcttca

SEQ ID NO: 11 - DNA, 887 bp, Homo sapiens
  misc_feature: cDNA sequence of human FCGR3A158F
tctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctc